(19) United States
(12) Patent Application Publication
(10) Pub. No.: US 2004/0116440 A1
(43) Pub. Date: Jun. 17, 2004

(54) Bombesin receptor antagonists

(76) Inventors: Michael Higginbottom, Sandwich (DE); Martyn Clive Pritchard, Sandwich (DE); Herman Thijs Stock, Sandwich (DE)

Correspondence Address: PFIZER INC., PATENT DEPARTMENT, MS8260-1611, EASTERN POINT ROAD, GROTON, CT, 06340, US

(21) Appl. No.: 10/416,779

(22) PCT Filed: Nov. 16, 2001
(86) PCT No.: PCT/EP01/14401

(30) Foreign Application Priority Data
Nov. 17, 2000 (GB) 0028104.8

(51) Int. Cl. A61K 31/506; A61K 31/497; A61K 31/501; A61K 31/4439; C 07D 4 3/02

(52) U.S. Cl. 514/252.03; 514/255.05; 514/256; 514/332; 514/338; 514/337; 514/340; 514/341; 514/342; 544/238; 544/333; 544/405; 546/255; 546/268.4; 546/269.4; 546/271.4; 546/272.1; 546/272.4; 546/272.7; 546/276.4; 546/280.4

(57) ABSTRACT

Bombesin receptor antagonists are provided which are useful for the diagnosis, prevention, or treatment of male sexual dysfunction in humans and animals, female sexual dysfunction in humans and animals, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus. The compounds of formula (I) or pharmaceutically acceptable salts thereof:

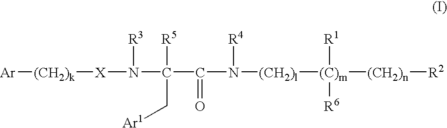

wherein k, l, m, n, X, Ar, Ar¹, R¹, R², R³, R⁴, R⁵ and R⁶ are as defined in the description.

### FIELD OF THE INVENTION

The present invention relates to chemical compounds that are bombesin receptor antagonists, to methods for the manufacture of the above compounds and to pharmaceutical compositions containing the above compounds. It also relates to the use of the above compounds in the manufacture of medicaments for the prophylaxis or treatment of a variety of disorders in animals (including humans). It further relates to methods for administration of the above compounds to patients for the prophylaxis or treatment of a variety of disorders.

### BACKGROUND TO THE INVENTION

Bombesin is a 14-amino acid peptide originally isolated from the skin of the European frog Bombina bombina (Anastasi A., et al., Experientia, 1971;27:166). It belongs to a class of peptides which share structural homology in their C-terminal decapeptide region (Dutta A. S., Small Peptides; Chemistry, Biology, and Clinical Studies, Chapter 2, pp 66-82). At present, two mammalian bombesin-like peptides have been identified (Battey J., etal., TINS, 1991;14:524), the decapeptide neuromedin B (NMB) and a 23-residue amino acid, gastrin-releasing peptide (GRP). Bombesin-like immunoreactivity has been detected in mammalian brain (Braun M., et al., Life. Sci., 1978;23:2721) and the GI tract (Walsh J. H., et al., Fed. Proc. Fed. Am. Soc. Exp. Biol., 1979;38:2315). This, together with studies measuring mRNA levels in rat brain (Battey J., et al., TINS, 1991;14:524), points to the widespread distribution of both NMB and GRP in mammalian peripheral and central nervous systems. NMB and GRP are believed to mediate a variety of biological actions via acting upon the corresponding bombesin receptors (for review, see WO 98/07718).

Bombesin evokes a number of central effects, e.g. feeding, scratching and peripheral effects e.g. contraction of rat oesophagus, secretion of gastrin, through actions at a heterogeneous population of receptors (for review, see Battey J. and Wada E., Trends Neurosci., 1991;14:524-528). The BB₁ receptor binds neuromedin B (NMB) with higher affinity than gastrin-related peptide (GRP) and neuromedin C (NMC) and BB₂ receptors bind GRP and NMC with greater affinity than NMB. More recently evidence has emerged of two more receptor subtypes denoted BB₃ and BB₄ but due to limited pharmacology, little is known, of their function at present. BB₁ and BB₂ receptors have a heterogeneous distribution within the central nervous system indicating that the endogenous ligands for these receptors may differentially modulate neurotransmission. Among other areas, BB₁ receptors are present in the ventromedial hypothalamus (Ladenheim E E et al, Brain Res., 1990; 537:233-240).

Both males and females can suffer from sexual dysfunction. Sexual dysfunctions are relatively common in the general population (see O'Donohue W, et al, Clin. Psychol. Rev. 1997;17:537-566). The disorder may relate to seeking sexual behaviour (proceptivity) and/or to acceptance of sexual behaviour, accompanied by sexual arousal (receptivity). The prevalence of sexual problems is higher in populations receiving medicaments, in particular antidepressants and, antihypertensives. A need for pharmacotherapy for sexual dysfunction is increasing, but there has been very little research effort directed at finding drugs to treat sexual dysfunction.

A component of male sexual dysfunction results from mechanical disorder(s), resulting in an inability to achieve penile erection or ejaculation. Treatment has been revolutionised by the unexpected discovery that cGMP PDE inhibitors, e.g. pyrazolo[4,3-d]pyrimidin-7-ones were useful in the treatment of erectile dysfunction and could be administered orally. One such compound that is currently being manufactured is sildenafil (Viagra). However, a second component of male sexual dysfunction is psychogenic disorders. Psychogenic disorders are also more prevalent in female sexual dysfunction. Thirty to 50% of American women complain of sexual dysfunction. Ageing, menopause, and decline in circulating oestrogen levels significantly increase the incidence of sexual complaints. In a recent publication (Berman J. R. et al. , Int. J. Impot. Res., 1999, 11: S31-38), the authors describe methodology for evaluating physiologic and subjective components of the female sexual response in the clinical setting and determine the effects of age and oestrogen status on them. In a further publication (Bonney R. C et al., Scrip's Complete Guide to Women's Healthcare, PJB Publications Ltd, London, 2000) the causes and management of female sexual dysfunction are discussed, including the use of tibolone (Livial), which is a synthetic steroid that mimics the effects of oestrogen and has been reported to have mild androgenic properties, and the use of testosterone.

WO 98/07718 discloses a class of non-peptide compounds capable of antagonizing the effects of NMB and/or GRP at bombesin receptors. The compounds are stated to be useful in treating or preventing a variety of disorders including depression, psychoses, seasonal affective disorders, cancer, feeding disorders, gastrointestinal disorders including colitis, Crohn's disease and inflammatory bowel disease, sleeping disorders, and memory impairment. U.S. Pat. No. 5,594,022 discloses non-peptide tachykinin antagonists expected to be useful in inflammatory disorders such as asthma and rheumatoid arthritis.

### SUMMARY OF THE INVENTION

We have surprisingly found a further class of bombesin receptor antagonists which are compounds of formula (I) or pharmaceutically acceptable salts thereof:

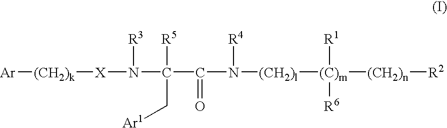

wherein:

k is 0, 1 or 2;

l is 0, 1, 2 or 3;

m is 0 or 1;

n is 0, 1 or 2;

X is —CO—, —OCO, —SO— or —SO₂—;

Ar is benzimidazolyl, benzofuryl, benzothiadiazolyl, benzothiazolyl, benzothienyl, benzopyrazinyl, benzotriazolyl, benzoxadiazolyl, furyl, imidazolyl, indanyl, indolyl, isoquinolyl, isoxazolyl, naphthyl, oxazolyl, phenyl, pyrazinyl, pyrazolyl, pyridyl, pyridazinyl, pyrimidyl, pyrrolyl, quinolyl, tetralinyl, tetrazolyl, thiazolyl, thienyl or triazolyl each unsubstituted or substituted with from 1 to 3 substituents selected from amino, acetyl, alkyl (straight chain or branched with from 1 to 6 carbon atoms), alkoxy, cyano, halogen, hydroxy, nitro, phenyl, pyridyl, pyrrolyl, isoxazolyl, phenoxy, tolyloxy, —CF₃, —OCF₃, —SO₂CF₃, —NHCONH₂, —CO₂H, —CH₂CO₂H, —CH₂CN, SO₂Me, SO₂NH₂, SO₂Ph, —(CH₂)_{q}NR⁷R⁸, —CONR⁹R¹⁰, and CO₂R¹¹, wherein q is 0, 1 or 2 and R⁷, R⁸, R⁹, R¹⁰, R¹¹ are each independently selected from hydrogen or straight or branched alkyl of up to 6 carbon atoms or cyclic alkyl of between 5 to 7 atoms which may contain 1 or 2 oxygen or nitrogen atoms or R⁷ and R⁸ or R⁹ and R¹⁰ together with the nitrogen atom to which they are linked can form a 5- to 7-membered aliphatic ring which may contain 1 or 2 oxygen or nitrogen atoms;

Ar¹ is independently selected from Ar and can also be pyridyl-N-oxide;

R¹ is hydrogen or straight or branched alkyl of up to 6 carbon atoms or cyclic alkyl of between 5 and 7 atoms which may contain 1 or 2 oxygen or nitrogen atoms;

R² is independently selected from Ar or is hydrogen, hydroxy, alkoxy, —NMe₂, —CONR¹²R¹³,

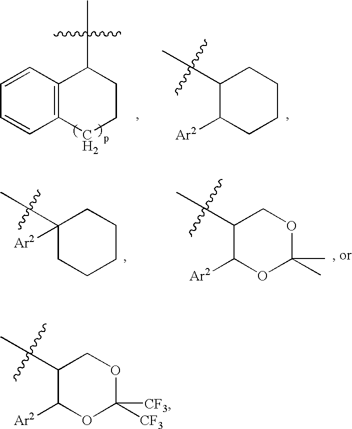

wherein p is 0, 1 or 2, Ar² is phenyl or pyridyl; and, R¹² and R¹³ are each independently selected from hydrogen, straight or branched alkyl of up to 6 carbon atoms or cyclic alkyl of between 5 and 7 carbon atoms;

R³, R⁴ and R⁵ are each independently selected from hydrogen and lower alkyl; and

R⁶ is hydrogen, methyl or forms with R¹ a ring of from 3 to 7 carbon atoms which can contain an oxygen or nitrogen atom, or R¹ and R⁶ can together be carbonyl;

provided that, when X is —OCO—, then l is 1, 2 or 3 and m is 1.

The compounds of the invention have been evaluated in receptor binding assays which measure their affinity in a cloned human NMB-preferring receptor (BB₁) assay and in a cloned human GRP-preferring receptor (BB₂) assay. It has been found that they have affinity for the BB₁ receptor and some of them also have affinity for the BB₂ receptor. Accordingly they may be useful for the diagnosis, prevention, or treatment of male sexual dysfunction in humans and animals, female sexual dysfunction in humans and animals, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus.

The invention further provides a method of antagonizing the effects of neuromedin B and/or gastrin-releasing peptide at bombesin receptors which comprises administering a compound of formula (I) to a patient.

The invention further provides a pharmaceutical composition comprising a therapeutically effective amount of a compound of formula (I) together with at least one pharmaceutically acceptable carrier or excipient.

The invention further provides a method for preventing or treating various diseases amenable to therapy by a bombesin receptor antagonist, including male or female sexual dysfunction, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus, said method comprising administering to a patient in need of such treatment an effective amount of a bombesin receptor antagonist of Formula (I).

The invention yet further provides the use of a compound of Formula (I) in the manufacture of a medicament for preventing or treating various diseases amenable to therapy by a bombesin receptor antagonist, including male or female sexual dysfunction, anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus.

### DESCRIPTION OF PREFERRED EMBODIMENTS

### Definitions

The compounds of Formula (I) are optically active. The scope of the invention therefore also includes:

All stereoisomers of the compounds of Formula (I).

Their solvates, hydrates and polymorphs (different crystalline lattice descriptors) of the compounds of Formula (I).

Pharmaceutical compositions of compounds of formula (I).

Prodrugs of the compounds of Formula (I) such as would occur to a person skilled in the art, see Bundgaard, et al., Acta Pharm. Suec., 1987;24:233-246.

The lower alkyl groups contemplated by the invention include straight or branched carbon chains of from 1 to 6 carbon atoms, except where specifically stated otherwise. They also include cycloalkyl groups, which are cyclic carbon chains having 3 to 7 carbon atoms, except where specifically stated otherwise, and which may be substituted with from 1 to 3 groups selected from halogens, nitro, straight or branched alkyl, and alkoxy.

The alkoxy groups contemplated by the invention comprise both straight and branched carbon chains of from 1 to 6 carbon atoms unless otherwise stated. Representative groups are methoxy, ethoxy, propoxy, i-propoxy, t-butoxy, and hexoxy.

The term “halogen” is intended to include fluorine, chlorine, bromine, iodine and astatine.

The term “amine” is intended to include free amino, alkylated amines, and acylated amines.

Optical Isomers and Salts

The compounds of Formula (I) all have at least one chiral centre and some have multiple chiral centres depending on their structure. In particular, the compounds of the present invention may exist as diastereomers, mixtures of diastereomers, or as the mixed or the individual optical enantiomers. The present invention contemplates all such forms of the compounds. The mixtures of diastereomers are typically obtained as a result of the reactions described more fully below. Individual diastereomers may be separated from mixtures of the diastereomers by conventional techniques such as column chromatography or repetitive recrystallization. Individual enantiomers may be separated by conventional methods well known in the art such as conversion to a salt with an optically active compound, followed by separation by chromatography or recrystallization and reconversion to the non-salt form.

Where it is appropriate to form a salt, the pharmaceutically acceptable salts include acetate, benzenesulfonate, benzoate, bicarbonate, bitartrate, bromide, calcium acetate, camsylate, carbonate, chloride, citrate, dihydrochloride, edetate, edisylate, estolate, esylate, fumarate, gluceptate, gluconate, glutamate, glycoloylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, mucate, napsylate, nitrate, pamoate (embonate), pantothenate, phosphate/diphosphate, polygalacturonate, salicylate, stearate, subacetate, succinate, sulfate, tannate, tartrate, theoclate, triethiodide, benzathine, chloroprocaine, choline, diethanolamine, ethylenediamine, meglumine, procaine, aluminum, calcium, lithium, magnesium, potassium; sodium, and zinc.

Preferred salts are made from strong acids. Such salts include hydrochloride, mesylate, and sulfate.

### Preferred Groups of Compounds

In a preferred group of the compounds of Formula (I),

k is 0 or 1;

l is 1;

m is 0 or 1;

n is 0 or 1;

X is —CO—, —OCO, or —SO₂—;

Ar is benzofuryl, furyl, indolyl, isoquinolyl, naphthyl, phenyl, pyridyl, quinolyl or thienyl each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ can form a ring of between 5 to 7 atoms which may contain 1 or 2 oxygen or nitrogen atoms, or R⁷ and R⁸ can be independently selected from hydrogen, straight or branched alkyl of up to 4 carbon atoms or cyclic alkyl of 5 carbon atoms;

Ar¹ is independently selected from Ar, preferably indolyl, and can also be pyridyl-N-oxide;

R¹ and R⁶ are cyclic alkyl of from 5 to 7 carbon atoms or R¹ and R⁶ together are carbonyl;

R² is independently selected from unsubstituted or substituted pyridyl or is hydrogen, hydroxy, alkoxy, —NMe₂, —CONR¹²R¹³ wherein R¹² and R¹³ are each independently selected from H and CH₃; and

R³, R⁴ and R⁵ are each independently selected from hydrogen and methyl.

In another preferred group of the compounds of Formula (I),

l is 1;

m is 1;

n is 0;

R² is 2-pyridyl;

R⁶ forms a cyclohexyl with R¹.

A particularly preferred group of compounds is of formula (Ia):

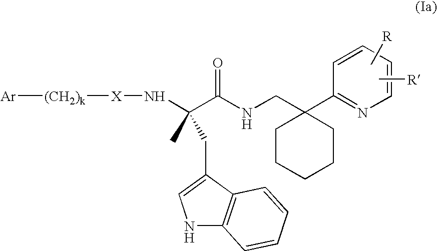

wherein Ar, k and X have the meanings given above at first, and the pyridine ring is optionally substituted by with 1 or 2 substituents, R and R′, independently selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ and R⁸ can be independently selected from hydrogen or cyclic alkyl of between 5 to 7 carbon atoms, and their pharmaceutically acceptable salts thereof.

In a further set of preferred compounds (Ia),

Ar is benzofuryl, furyl, indolyl, isoquinolyl, naphthyl, phenyl, pyridyl, quinolyl or thienyl, each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ or R⁸ can be a independently selected from hydrogen or cyclic alkyl of 5 carbon atoms, and

X is —CO—, —OCO— or —SO₂.

Preferred N-terminal Amide Derivatives

Amongst N-terminal amide derivatives (Compounds of formula I, wherein X is —CO—) the following compounds are most preferred:

N-{(S)-2-(1H-indol-3-yl)-1-methyl-1-[(1-pyridin-2-yl-cyclohexylmethyl)-carbamoyl]-ethyl}--4-nitro-benzamide;

C-dimethylamino-N-{(S)-2-(1H-indol-3-yl)-1-methyl-1-[(1-pyridin-2-yl-cyclohexylmethyl)-carbamoyl]-ethyl}-benzamide;

1H-indole-2-carboxylic acid {(S)-2-(1H-indol-3-yl)-1-methyl-1-[(1-pyridin-2-yl-cyclohexylmethyl)-carbamoyl]-ethyl}-amide;

benzo[b]thiophene-2-carboxylic acid {(S)-2-(1H-indol-3-yl)-1-methyl-1-[(1-pyridin-2-yl-cyclohexylmethyl)-carbamoyl]-ethyl}-amide;

1H-indole-5-carboxylic acid {(S)-2-(1H-indol-3-yl)-1-methyl-1-[(1-pyridin-2-yl-cyclohexylmethyl)-carbamoyl]-ethyl}-amide; and

1H-indole-2-carboxylic acid ((S)-2-(1H-indol-3-yl)-1-{[1-(5-methoxy-pyridin-2-yl)-cyclohexylmethyl]-carbamoyl}-1-methyl-ethyl )-amide.

Other preferred N-terminal amide derivatives include the following:

N-{(S)-2-(1H-indol-3-yl)-1-methyl-1-[(1-pyridin-2-yl-cyclohexylmethyl)-carbamoyl]-ethyl}-benzamide;

N-{(S)-2-(1H-indol-3-yl)-1-methyl-1-[(1-pyridin-2-yl-cyclohexylmethyl)-carbamoyl]-ethyl}-4-methyl-benzamide;

4-chloro-N-{(S)-2-(1H-indol-3-yl)-1-methyl-1-[(1-pyridin-2-yl-cyclohexylmethyl)-carbamoyl]-ethyl}-benzamide;

N-{(S)-2-(1H-indol-3-yl)-1-methyl-1-[(1-pyridin-2-yl-cyclohexylmethyl)-carbamoyl]-ethyl}-4-methoxy-benzamide;

N-{(S)-2-(1H-indol-3-yl)-1-methyl-1-[(1-pyridin-2-yl-cyclohexylmethyl)-carbamoyl]-ethyl}-4-methanesulfonyl-benzamide;

3-cyano-N-{(S)-2-(1H-indol-3-yl)-1-methyl-1-[(1-pyridin-2-yl-cyclohexylmethyl)-carbamoyl]-ethyl}-benzamide;

3-chloro-N-{(S)-2-(1H-indol-3-yl)-1-methyl-1-[(1-pyridin-2-yl-cyclohexylmethyl)-carbamoyl]-ethyl}-benzamide;

N-{(S)-2-(1H-indol-3-yl)-1-methyl-1-[(1-pyridin-2-yl-cyclohexylmethyl)-carbamoyl]-ethyl}-3-methoxy-benzamide;

N-{(S)-2-(1H-indol-3-yl)-1-methyl-1-[(1-pyridin-2-yl-cyclohexylmethyl)-carbamoyl]-ethyl}-3-methanesulfonyl-benzamide;

dimethylamino-N-{(S)-2-(1H-indol-3-yl)-1-methyl-1-[(1-pyridin-2-yl-cyclohexylmethyl)-carbamoyl]-ethyl}-benzamide;

N-{(S)-2-(1H-indol-3-yl)-1-methyl-1-[(1-pyridin-2-yl-cyclohexylmethyl)-carbamoyl]-ethyl}-3-methyl-benzamide;

2-chloro-N-{(S)-2-(1H-indol-3-yl)-1-methyl-1-[(1-pyridin-2-yl-cyclohexylmethyl)-carbamoyl]-ethyl}-benzamide;

N-{(S)-2-(1H-indol-3-yl)-1-methyl-1-[(1-pyridin-2-yl-cyclohexylmethyl)-carbamoyl]-ethyl}-2-nitro-benzamide;

N-{(S)-2-(1H-indol-3-yl)-1-methyl-1-[(1-pyridin-2-yl-cyclohexylmethyl)-carbamoyl]-ethyl}-2-methoxy-benzamide;

N-{(S)-2-(1H-indol-3-yl)-1-methyl-1-[(1-pyridin-2-yl-cyclohexylmethyl)-carbamoyl]-ethyl}-2-methyl-benzamide;

2-fluoro-N-{(S)-2-(1H-indol-3-yl)-1-methyl-1-[(1-pyridin-2-yl-cyclohexylmethyl)-carbamoyl]-ethyl}-benzamide;

(S)-3-(1H-indol-3-yl)-2-methyl-N-(1-pyridin-2-yl-cyclohexylmethyl)-2-(2-p-tolyl-ethanoylamino)-propionamide;

(S)-3-(1H-indol-3-yl)-2-methyl-N-(1-pyridin-2-yl-cyclohexylmethyl)-2-(2-o-tolyl-ethanoylamino)-propionamide;

(S)-2-[2-(4-hydroxy-phenyl)-ethanoylamino]-3-(1H-indol-3-yl)-2-methyl-N-(1-pyridin-2-yl-cyclohexylmethyl)-propionamide;

(S)-2-[2-(3-hydroxy-phenyl)-ethanoylamino]-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(2-m-tolyl-ethanoylamino)-propionamide;

(S)-2-[2-(2-fluoro-phenyl)-ethanoylamino]-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(2-thiophen-3-yl-ethanoylamino)-propionamide;

N-{(S)-2-(1H-indol-3-yl)-1-methyl-1-[(1-pyridin-2-yl-cyclohexylmethyl)-carbamoyl]-ethyl}-isonicotinamide;

furan-3-carboxylic acid {(S)-2-(1H-indol-3-yl)-1-methyl-1-[(1-pyridin-2-yl-cyclohexylmethyl)-carbamoyl]-ethyl}-amide;

furan-2-carboxylic acid {(S)-2-(1H-indol-3-yl)-1-methyl-1-[(1-pyridin-2-yl-cyclohexylmethyl)-carbamoyl]-ethyl}-amide;

5-methyl-isoxazole-3-carboxylic acid {(S)-2-(1H-indol-3-yl)-1-methyl-1-[(1-pyridin-2-yl-cyclohexylmethyl)-carbamoyl]-ethyl}-amide;

1-methyl-1H-pyrrole-2-carboxylic acid {(S)-2-(1H-indol-3-yl)-1-methyl-1-[(1-pyridin-2-yl-cyclohexylmethyl)-carbamoyl]-ethyl}-amide;

thiophene-2-carboxylic acid {(S)-2-(1H-indol-3-yl)-1-methyl-1-[(1-pyridin-2-yl-cyclohexylmethyl)-carbamoyl]-ethyl}-amide;

thiophene-3-carboxylic acid {(S)-2-(1H-indol-3-yl)-1-methyl-1-[(1-pyridin-2-yl-cyclohexylmethyl)-carbamoyl]-ethyl}-amide;

1H-indole-6-carboxylic acid {(S)-2-(1H-indol-3-yl)-1-methyl-1-[(1-pyridin-2-yl-cyclohexylmethyl)-carbamoyl]-ethyl}-amide;

1H-indole-5-carboxylic acid {(S)-2-(1H-indol-3-yl)-1-methyl-1-[(1-pyridin-2-yl-cyclohexylmethyl)-carbamoyl]-ethyl}-amide;

1H-indole-4-carboxylic acid {(S)-2-(1H-indol-3-yl)-1-methyl-1-[(1-pyridin-2-yl-cyclohexylmethyl)-carbamoyl]-ethyl}-amide;

1H-indole-7-carboxylic acid {(S)-2-(1H-indol-3-yl)-1-methyl-1-[(1-pyridin-2-yl-cyclohexylmethyl)-carbamoyl]-ethyl}-amide;

1-methyl-1H-indole-2-carboxylic acid {(S)-2-(1H-indol-3-yl)-1-methyl-1-[(1-pyridin-2-yl-cyclohexylmethyl)-carbamoyl]-ethyl}-amide;

benzothiazole-6-carboxylic acid {(S)-2-(1H-indol-3-yl)-1-methyl-1-[(1-pyridin-2-yl-cyclohexylmethyl)-carbamoyl]-ethyl}-amide;

1H-benzotriazole-5-carboxylic acid {(S)-2-(1H-indol-3-yl)-1-methyl-1-[(1-pyridin-2-yl-cyclohexylmethyl)-carbamoyl]-ethyl}-amide;

3-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;

5-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;

6-methyl-pyridine-2-carboxylic acid {(S)-2-(1H-indol-3-yl)-1-methyl-1-[(1-pyridin-2-yl-cyclohexylmethyl)-carbamoyl]-ethyl}-amide;

isoquinoline-3-carboxylic acid {(S)-2-(1H-indol-3-yl)-1-methyl-1-[(1-pyridin-2-yl-cyclohexylmethyl)-carbamoyl]-ethyl}-amide;

quinoxaline-2-carboxylic acid {(S)-2-(1H-indol-3-yl)-1-methyl-1-[(1-pyridin-2-yl-cyclohexylmethyl)-carbamoyl]-ethyl}-amide;

quinoline-8-carboxylic acid {(S)-2-(1H-indol-3-yl)-1-methyl-1-[(1-pyridin-2-yl-cyclohexylmethyl)-carbamoyl]-ethyl}-amide;

5-phenyl-oxazole-4-carboxylic acid {(S)-2-(1H-indol-3-yl)-1-methyl-1-[(1-pyridin-2-yl-cyclohexylmethyl)-carbamoyl]-ethyl}-amide;

(S)-3-(1H-indol-3-yl)-2-[2-(4-methoxy-phenyl)-ethanoylamino]-2-methyl-N-(1-pyridin-2-yl-cyclohexylmethyl)-propionamide;

(S)-2-[2-(4-dimethylamino-phenyl)-ethanoylamino]-3-(1H-indol-3-yl)-2-methyl-N-(1-pyridin-2-yl-cyclohexylmethyl)-propionamide;

(S)-3-(1H-indol-3-yl)-2-methyl-2-[2-(2-nitro-phenyl)-ethanoylamino]-N-(1-pyridin-2-yl-cyclohexylmethyl)-propionamide;

(S)-3-(1H-indol-3-yl)-2-[2-(2-methoxy-phenyl)-ethanoylamino]-2-methyl-N-(1-pyridin-2-yl-cyclohexylmethyl)-propionamide; and

N-{(S)-2-(1H-indol-3-yl)-1-methyl-1-[(1-pyridin-2-yl-cyclohexylmethyl)-carbamoyl]-ethyl}-2-pyrrol-1-yl-benzamide.

Preferred N-terminal Urethane Derivatives

Amongst N-terminal urethane derivatives (Compounds of formula I wherein X is —OC(═O)—) the following compounds are particularly preferred:

{(S)-2-(1H-indol-3-yl)-1-methyl-1-[(1-pyridin-2-yl-cyclohexylmethyl)-carbamoyl]-ethyl}-carbamic acid naphthalen-1-ylmethyl ester;

{(S)-2-(1H-indol-3-yl)-1-methyl-1-[(1-pyridin-2-yl-cyclohexylmethyl)-carbamoyl]-ethyl}-carbamic acid 3,4-dichloro-benzyl ester;

{(S)-2-(1H-indol-3-yl)-1-methyl-1-[(1-pyridin-2-yl-cyclohexylmethyl)-carbamoyl]-ethyl}-carbamic acid 3-nitro-benzyl ester;

{(S)-2-(1H-indol-3-yl)-1-methyl-1-[(1-pyridin-2-yl-cyclohexylmethyl)-carbamoyl]-ethyl}-carbamic acid 3-trifluoromethyl-benzyl ester;

{(S)-2-(1H-indol-3-yl)-1-methyl-1-[(1-pyridin-2-yl-cyclohexylmethyl)-carbamoyl]-ethyl}-carbamic acid quinolin-6-ylmethyl ester;

{(S)-2-(1H-indol-3-yl)-1-methyl-1-[(1-pyridin-2-yl-cyclohexylmethyl)-carbamoyl]-ethyl}-carbamic acid 4-nitro-benzyl ester; and

{(S)-2-(1H-indol-3-yl)-1-methyl-1-[(1-pyridin-2-yl-cyclohexylmethyl)-carbamoyl]-ethyl}-carbamic acid 3-cyano-benzyl ester.

Other preferred N-terminal urethane derivatives include the following:

{(S)-2-(1H-indol-3-yl)-1-methyl-1-[(1-pyridin-2-yl-cyclohexylmethyl)-carbamoyl]-ethyl}-carbamic acid 3,4-dimethoxy-benzyl ester;

{(S)-2-(1H-indol-3-yl)-1-methyl-1-[(1-pyridin-2-yl-cyclohexylmethyl)-carbamoyl]-ethyl}-carbamic acid naphthalen-2-ylmethyl ester;

{(S)-2-(1H-indol-3-yl)-1-methyl-1-[(1-pyridin-2-yl-cyclohexylmethyl)-carbamoyl]-ethyl}-carbamic acid indan-2-yl ester;

{(S)-2-(1H-indol-3-yl)-1-methyl-1-[(1-pyridin-2-yl-cyclohexylmethyl)-carbamoyl]-ethyl}-carbamic acid 4-methoxy-benzyl ester;

{(S)-2-(1H-indol-3-yl)-1-methyl-1-[(1-pyridin-2-yl-cyclohexylmethyl)-carbamoyl]-ethyl}-carbamic acid 4-chloro-benzyl ester;

{(S)-2-(1H-indol-3-yl)-1-methyl-1-[(1-pyridin-2-yl-cyclohexylmethyl)-carbamoyl]-ethyl}-carbamic acid 2-fluoro-benzyl ester;

{(S)-2-(1H-indol-3-yl)-1-methyl-1-[(1-pyridin-2-yl-cyclohexylmethyl)-carbamoyl]-ethyl}-carbamic acid 2-chloro-benzyl ester;

{(S)-2-(1H-indol-3-yl)-1-methyl-1-[(1-pyridin-2-yl-cyclohexylmethyl)-carbamoyl]-ethyl}-carbamic acid 4-nitro-benzyl ester;

{(S)-2-(1H-indol-3-yl)-1-methyl-1-[(1-pyridin-2-yl-cyclohexylmethyl)-carbamoyl]-ethyl}-carbamic acid 2-methyl-benzyl ester;

{(S)-2-(1H-indol-3-yl)-1-methyl-1-[(1-pyridin-2-yl-cyclohexylmethyl)-carbamoyl]-ethyl}-carbamic acid 4-tert-butyl-benzyl ester;

{(S)-2-(1H-indol-3-yl)-1-methyl-1-[(1-pyridin-2-yl-cyclohexylmethyl)-carbamoyl]-ethyl}-carbamic acid 2-methoxy-benzyl ester;

{(S)-2-(1H-indol-3-yl)-1-methyl-1-[(1-pyridin-2-yl-cyclohexylmethyl)-carbamoyl]-ethyl}-carbamic acid 4-trifluoromethyl-benzyl ester;

{(S)-2-(1H-indol-3-yl)-1-methyl-1-[(1-pyridin-2-yl-cyclohexylmethyl)-carbamoyl]-ethyl}-carbamic acid 3-ethoxy-benzyl ester;

{(S)-2-(1H-indol-3-yl)-1-methyl-1-[(1-pyridin-2-yl-cyclohexylmethyl)-carbamoyl]-ethyl}-carbamic acid 3-cyano-benzyl ester;

{(S)-2-(1H-indol-3-yl)-1-methyl-1-[(1-pyridin-2-yl-cyclohexylmethyl)-carbamoyl]-ethyl}-carbamic acid 2,4-dichloro-benzyl ester;

{(S)-2-(1H-indol-3-yl)-1-methyl-1-[(1-pyridin-2-yl-cyclohexylmethyl)-carbamoyl]-ethyl}-carbamic acid 3-methyl-benzyl ester;

{(S)-2-(1H-indol-3-yl)-1-methyl-1-[(1-pyridin-2-yl-cyclohexylmethyl)-carbamoyl]-ethyl}-carbamic acid 3-phenoxy-benzyl ester;

{(S)-2-(1H-indol-3-yl)-1-methyl-1-[(1-pyridin-2-yl-cyclohexylmethyl)-carbamoyl]-ethyl}-carbamic acid 4-methyl-benzyl ester; and

{(S)-2-(1H-indol-3-yl)-1-methyl-1-[(1-pyridin-2-yl-cyclohexylmethyl)-carbamoyl]-ethyl}-carbamic acid 2,3-dichloro-benzyl ester.

Preferred N-terminal Sulfonamide Derivatives

Amongst N-terminal sulfonamide derivatives (compounds of formula I, wherein X is —SO₂—) the following compounds are particularly preferred:

(S)-3-(1H-indol-3-yl)-2-methyl-2-phenylmethanesulfonylamino-N-(1-pyridin-2-yl-cyclohexylmethyl)-propionamide;

(S)-2-(2-chloro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(naphthalene-1-sulfonylamino)-N-(1-pyridin-2-yl-cyclohexylmethyl)-propionamide;

(S)-3-(1H-indol-3-yl)-2-methyl-N-(1-pyridin-2-yl-cyclohexylmethyl)-2-(quinoline-8-sulfonylamino)-propionamide;

(S)-3-(1H-indol-3-yl)-2-methyl-N-(1-pyridin-2-yl-cyclohexylmethyl)-2-(2-trifluoromethyl-benzenesulfonylamino)-propionamide;

(S)-2-(biphenyl-2-sulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(5-methyl-2-phenoxy-benzenesulfonyl-amino)-N-(1-pyridin-2-yl-cyclohexylmethyl)-propionamide; and

(S)-3-(1H-indol-3-yl)-2-methyl-N-(1-pyridin-2-yl-cyclohexylmethyl)-2-(2-p-tolyloxy-benzenesulfonylamino)-propionamide.

Further preferred N-terminal sulfonamide derivatives include the following:

(S)-3-(1H-indol-3-yl)-2-methyl-N-(1-pyridin-2-yl-cyclohexylmethyl)-2-(toluene-4-sulfonylamino)-propionamide;

(S)-3-(1H-indol-3-yl)-2-methanesulfonylamino-2-methyl-N-(1-pyridin-2-yl-cyclohexylmethyl)-propionamide;

(S)-2-(2-fluoro-benzenesulfonylamino)-3-(1H-indol-3-yl)-2-methyl-N-(1-pyridin-2-yl-cyclohexylmethyl)-propionamide;

(S)-2-(4-chloro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(2,2,2-trifluoro-ethanesulfonylamino)-propionamide;

(S)-2-(5-dimethylamino-naphthalene-1-sulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(naphthalene-2-sulfonylamino)-N-(1-pyridin-2-yl-cyclohexylmethyl)-propionamide;

(S)-3-(1H-indol-3-yl)-2-methyl-N-(1-pyridin-2-yl-cyclohexylmethyl)-2-(thiophene-2-sulfonylamino)-propionamide;

(S)-3-(1H-indol-3-yl)-2-methyl-2-(3-nitro-benzenesulfonylamino)-N-(1-pyridin-2-yl-cyclohexylmethyl)-propionamide;

(S)-2-(4-fluoro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(4-nitro-benzenesulfonylamino)-N-(1-pyridin-2-yl-cyclohexylmethyl)-propionamide;

(S)-3-(1H-indol-3-yl)-2-methyl-N-(1-pyridin-2-yl-cyclohexylmethyl)-2-(3-trifluoromethyl-benzenesulfonylamino)-propionamide;

(S)-2-(3,4-dichloro-benzenesulfonylamino)-3-(1H-indol-3-yl)-2-methyl-N-(1-pyridin-2-yl-cyclohexylmethyl)-propionamide;

(S)-2-(3-fluoro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(4-trifluoromethyl-benzenesulfonylamino)-propionamide;

(S)-2-(5-chloro-thiophene-2-sulfonylamino)-3-(1H-indol-3-yl)-2-methyl-N-(1-pyridin-2-yl-cyclohexylmethyl)-propionamide;

(S)-2-(3-chloro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(toluene-3-sulfonylamino)-propionamide;

(S)-2-(3,4-dimethoxy-benzenesulfonylamino)-3-(1H-indol-3-yl)-2-methyl-N-(1-pyridin-2-yl-cyclohexylmethyl)-propionamide;

(S)-2-(4-cyano-benzenesulfonylamino)-3-(1H-indol-3-yl)-2-methyl-N-(1-pyridin-2-yl-cyclohexylmethyl)-propionamide;

(S)-2-(2-cyano-benzenesulfonylamino)-3-(1H-indol-3-yl)-2-methyl-N-(1-pyridin-2-yl-cyclohexylmethyl)-propionamide;

(S)-2-(5-chloro-1,3-dimethyl-1H-pyrazole-4-sulfonylamino)-3-(1H-indol-3-yl)-2-methyl-N-(1-pyridin-2-yl-cyclohexylmethyl)-propionamide;

(S)-2-(3,5-dimethyl-isoxazole-4-sulfonylamino)-3-(1H-indol-3-yl)-2-methyl-N-(1-pyridin-2-yl-cyclohexylmethyl)-propionamide;

(S)-2-(benzo[1,2,5]thiadiazole-4-sulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(1-methyl-1H-imidazole-4-sulfonylamino)-N-(1-pyridin-2-yl-cyclohexylmethyl)-propionamide;

(S)-2-(benzo[1,2,5]oxadiazole-4-sulfonylamino)-3-(1H-indol-3-yl)-2-methyl-N-(1-pyridin-2-yl-cyclohexylmethyl)-propionamide;

3-{(S)-2-(1H-indol-3-yl)-1-methyl-1-[(1-pyridin-2-yl-cyclohexylmethyl)-carbamoyl]-ethylsulfamoyl)-thiophene-2-carboxylic acid methyl ester;

(S)-3-(1H-indol-3-yl)-2-(5-isoxazol-3-yl-thiophene-2-sulfonylamino)-2-methyl-N-(1-pyridin-2-yl-cyclohexylmethyl)-propionamide;

(S)-3-(1H-indol-3-yl)-2-methyl-2-(2-nitro-phenylmethanesulfonylamino)-N-(1-pyridin-2-yl-cyclohexylmethyl)-propionamide;

(S)-2-(3-cyano-benzenesulfonylamino)-3-(1H-indol-3-yl)-2-methyl-N-(1-pyridin-2-yl-cyclohexylmethyl)-propionamide;

(S)-2-(1,2-dimethyl-1H-imidazole-4-sulfonylamino)-3-(1H-indol-3-yl)-2-methyl-N-(1-pyridin-2-yl-cyclohexylmethyl)-propionamide;

(S)-3-(1H-indol-3-yl)-2-(3-methoxy-benzenesulfonylamino)-2-methyl-N-(1-pyridin-2-yl-cyclohexylmethyl)-propionamide;

(S)-3-(1H-indol-3-yl)-2-methyl-2-(8-nitro-naphthalene-1-sulfonylamino)-N-(1-pyridin-2-yl-cyclohexylmethyl)-propionamide;

(S)-2-(2-chloro-5-nitro-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(2,4,6-trichloro-benzenesulfonylamino)-propionamide;

(S)-2-(4-chloro-2-nitro-benzenesulfonylamino)-3-(1H-indol-3-yl)-2-methyl-N-(1-pyridin-2-yl-cyclohexylmethyl)-propionamide;

(S)-2-(5-benzenesulfonyl-thiophene-2-sulfonylamino)-3-(1H-indol-3-yl)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(4-trifluoromethoxy-benzenesulfonylamino)-propionamide;

(S)-3-(1H-indol-3-yl)-2-methyl-2-(5-methyl-2-phenoxy-benzenesulfonylamino)-N-(1-pyridin-2-yl-cyclohexylmethyl)-propionamide;

(S)-3-(1H-indol-3-yl)-2-methyl-N-(1-pyridin-2-yl-cyclohexylmethyl)-2-(2-p-tolyloxy-benzenesulfonylamino)-propionamide;

2-{(S)-2-(1H-indol-3-yl)-1-methyl-1-[(1-pyridin-2-yl-cyclohexylmethyl)-carbamoyl]-ethylsulfamoyl}-benzoic acid methyl ester;

(S)-2-(3-chloro-4-fluoro-benzenesulfonylamino)-3-(1H-indol-3-yl)-2-methyl-N-(1-pyridin-2-yl-cyclohexylmethyl)-propionamide;

(S)-2-(2,5-dichloro-thiophene-3-sulfonylamino)-3-(1H-indol-3-yl)-2-methyl-N-(1-pyridin-2-yl-cyclohexylmethyl)-propionamide;

(S)-2-(3-chloro-4-methyl-benzenesulfonylamino)-3-(1H-indol-3-yl)-2-methyl-N-(1-pyridin-2-yl-cyclohexylmethyl)-propionamide;

(S)-3-(1H-indol-3-yl)-2-(2-methoxy-4-methyl-benzenesulfonylamino)-2-methyl-N-(1-pyridin-2-yl-cyclohexylmethyl)-propionamide;

(S)-3-(1H-indol-3-yl)-2-methyl-N-(1-pyridin-2-yl-cyclohexylmethyl)-2-(5-pyridin-2-yl-thiophene-2-sulfonylamino)-propionamide;

(S)-2-(5-bromo-6-chloro-pyridine-3-sulfonylamino)-3-(1H-indol-3-yl)-2-methyl-N-(1-pyridin-2-yl-cyclohexylmethyl)-propionamide;

(S)-2-(2,4-dinitro-benzenesulfonylamino)-3-(1H-indol-3-yl)-2-methyl-N-(1-pyridin-2-yl-cyclohexylmethyl)-propionamide;

(S)-3-(1H-indol-3-yl)-2-(4-methanesulfonyl-benzenesulfonylamino)-2-methyl-N-(1-pyridin-2-yl-cyclohexylmethyl)-propionamide;

(S)-2-(4-tert-butyl-benzenesulfonylamino)-3-(1H-indol-3-yl)-2-methyl-N-(1-pyridin-2-yl-cyclohexylmethyl)-propionamide;

(S)-2-(2,4-dichloro-5-methyl-benzenesulfonylamino)-3-(1H-indol-3-yl)-2-methyl-N-(1-pyridin-2-yl-cyclohexylmethyl)-propionamide;

(S)-2-(2-chloro-5-trifluoromethyl-benzenesulfonylamino)-3-(1H-indol-3-yl)-2-methyl-N-(1-pyridin-2-yl-cyclohexylmethyl)-propionamide;

(S)-3-(1H-indol-3-yl)-2-methyl-2-(2-nitro-4-trifluoromethyl-benzenesulfonyl-amino)-N-(1-pyridin-2-yl-cyclohexylmethyl)-propionamide; and

(S)-2-(4-butyl-benzenesulfonylamino)-3-(1H-indol-3-yl)-2-methyl-N-(1-pyridin-2-yl-cyclohexylmethyl)-propionamide.

### Preparative Methods

Compounds of the Formula (I) in which X is —CO— can be prepared by condensing an acid of the Formula (II)

Ar—(CH₂)ₖ—COOH (II)

or a derivative thereof with an amine of the formula (III)

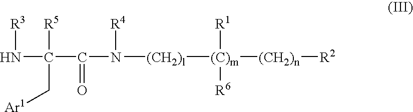

in an aprotic polar solvent in the presence of an appropriate catalyst, the values of the substituents Ar, Ar¹ and R¹ to R⁶ and the parameters k to n being as defined above with reference to formula (I), and optionally converting the resulting product to a pharmaceutically acceptable salt. For example, the condensation may be carried out in DMF using O-benzotriazol-1-yl-N,N,N′,N′-tetramethyluronium hexafluorophosphate (HBTU) and N,N-diisopropyl-ethylamine (DIPEA) as catalyst.

Compounds of the Formula (I) in which X is —O(C═O)— can be prepared by forming a carbonate from an alcohol of the Formula (IV)

Ar—(CH₂)ₖ—OH (IV)

and reacting the carbonate with an amine of the Formula (III)

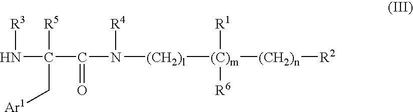

in an aprotic polar solvent in the presence of a base, the values of the substituents Ar, Ar¹ and R¹ to R⁶ and the parameters k to n being as defined above with reference to Formula (I), and optionally converting the resulting product to a pharmaceutically acceptable salt. For example, the compound of Formula (IV) may be reacted with 4-nitrophenyl chloroformate in dichloromethane using pyridine as catalyst, and the resulting carbonate may be reacted with the amine of Formula (III) in dimethyl formamide using N,N-dimethyl-4-amino pyridine as catalyst.

Compounds of the Formula (I) in which X is —SO₂— can be prepared by condensing a sulfonyl chloride of the Formula (V)

Ar—(CH₂)ₖ—SO₂Cl (V)

with an amine of the Formula (III)

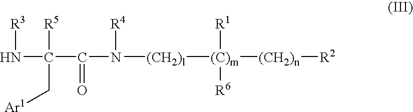

in an aprotic polar solvent in the presence of a base as catalyst, the values of the substituents Ar, Ar¹ and R¹ to R⁶ and the parameters k to n being as defined above with reference to Formula (I), and optionally converting the resulting product to a pharmaceutically acceptable salt. For example, the condensation may be carried out in DMF in the presence of N,N-diisopropylethylamine and N,N-dimethyl-4-aminopyridine.

In the above methods, the amine of Formula (111) is preferably a chiral amine of Formula (VI)

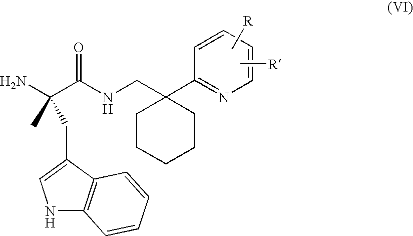

wherein the pyridine ring is optionally substituted by with 1 or 2 substituents R and R′ selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷and R⁸ can be independently selected from hydrogen or cyclic alkyl of between 5 to 7 carbon atoms, methoxy being a particularly preferred substituent, as in the chiral amine (VIb):

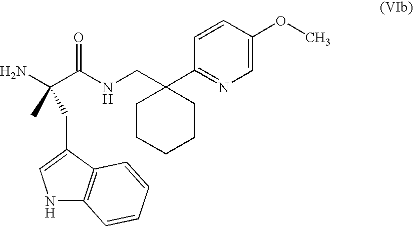

This intermediate (VIb), which is (S)-2-amino-3-(1H-indol-3-yl)-N-[1-(5-methoxy-pyridin-2-yl)-cyclohexylmethyl]-2-methyl-propionamide, is novel.

Pharmaceutical Compositions

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can comprise one or more substances that may also act as diluents, flavoring agents, solubilizers, lubricants, suspending agents, binders, or tablet disintegrating agents; it can also be an encapsulating material. In powders, the carrier is a finely divided solid that is in a mixture with the finely divided active component. In tablets, the active component is mixed with the carrier having the necessary binding properties in suitable proportions and compacted in the shape and size desired. The powders and tablets preferably contain 5% to about 70% of the active component. Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid form preparations include solutions, suspensions, and emulsions. Sterile water or water-propylene glycol solutions of the active compounds may be mentioned as an example of liquid preparations suitable for parenteral administration. Liquid preparations can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Preferably the pharmaceutical preparation is in unit dosage form. In such form, the preparation is divided into unit doses containing appropriate quantities of the active component. The unit dosage form can be a packaged preparation, the package containing discrete quantities of the preparation, for example, packeted tablets, capsules, and powders in vials or ampoules. The unit dosage form can also be a capsule, cachet, or tablet itself, or it can be the appropriate number of any of these packaged forms.

For preparing suppository preparations, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

The dosage can range from about 0.1 mmol/kg of active compound per kg of body weight to about 500 mmol/kg body weight. A preferred dosage is about 5 to about 50 mmol of active compound per kg of body weight.

Sexual Dysfunction

Although there is no known direct link between the effects of bombesin receptor ligands and sexual function, the presence of receptors in hypothalamic areas might suggest a neuromodulatory effect on functions controlled at a hypothalamic level, and these could include, among others, feeding and sexual behaviour.

Female sexual dysfunction can be grouped into four classes (Scrip's Complete Guide to Women's Healthcare, p.194-205, April 2000), which include hypoactive sexual desire disorders, sexual arousal disorders, orgasmic disorders or anorgasmy and sexual pain disorders. Hypoactive sexual desire disorders can be characterized as persistent or recurrent lack of sexual thoughts/fantasies and lack of receptivity to sexual activity, causing personal distress. Common problems include sexual aversion disorders. Sexual arousal disorders can be characterized as persistent or recurrent inability to achieve or maintain adequate sexual excitement, causing personal distress. Common problems include lack of or diminished vaginal lubrication, decreased clitoral and labial sensation, decreased clitoral and labial engorgement and lack of vaginal smooth muscle relaxation. Orgasmic disorders can be characterized as persistent or recurrent difficulty or delay in attaining orgasm after adequate sexual stimulation and arousal, causing personal distress. Sexual pain disorders can be characterized by dyspareunia, (characterised by recurrent or persistent genital pain associated with sexual intercourse), vaginismus (characterised by recurrent or persistent involuntary spasm of the muscles of the outer third of the vagina which interferes with vaginal penetration, causing personal distress) and other pain disorders (characterised by recurrent or persistent genital pain induced by non coital sexual stimulation).

The compounds of this invention are useful in the treatment of female sexual dysfunction, and this includes female sexual dysfunction associated with hypoactive sexual desire disorders, sexual arousal disorders, orgasmic disorders or anorgasmy, or sexual pain disorders.

The psychogenic component of male sexual dysfunction has been classified by the nomenclature committee of the International Society for Impotence Research (and is illustrated in Sachs B. D., Neuroscience and Biobehavioral Review 24: 541-560, 2000) as generalised type, characterised by a general unresponsiveness or primary lack of sexual arousal, and ageing-related decline in sexual arousability, characterised by generalised inhibition or chronic disorders of sexual intimacy. The inventors believe that there are common mechanisms underlying the pathologies of male and female phychogenic sexual dysfunctions.

The compounds of this invention are useful in the treatment of male sexual dysfunction, especially drug induced sexual dysfunction psychogenic male sexual dysfunction associated with generalised unresponsiveness and ageing-related decline in sexual arousability.

Anxiety, Panic Attacks and Social Phobia

Anxiety is a very commonly observed symptom, for which benzodiazepines are the primary treatment agents. Chlordiazepoxide, diazepam, oxazepam, lorazepam, prazepam and alprazolam are most commonly used for this purpose in the United States. However anxiolytic benzodiazepines may also cause Sedation, they have muscle-relaxant, sedative-hypnotic, and amnestic side effects; they also tend to potentiate the effects of alcohol. Some tolerance to their effects may develop, withdrawal after chronic use frequently induces rebound anxiety, and long-term use of benzodiazepines, particularly with escalating doses, can lead to dependence. Therefore there is a need for anxiolytic treatments with a reduced dependence liability.

Recent findings suggest a role of bombesin-like peptides in stress and anxiety (Plamondon H. et al. (1996) Soc. Neurosci. 22: Abstract 181.13): antisense oligonucleotides to mRNA for GRP receptors and NMB receptors were infused i.c.v. in rats over 2 days, resulting in a reduction of bombesin binding site density in the brain, as measured by receptor autoradiography. Rats treated with the antis ense oligonucleotides spent significantly more time on the anxiogenic fields of an elevated plus maze, or of a trough-tunnel oval maze, reflecting an anxiolytic effect of treatment, as compared to control animals.

The compounds of the instant invention are useful in the treatment of anxiety, panic attacks and social phobia.

Depression

The compounds of the invention are useful in the treatment of depression. The following publication provides evidences of the role of bombesin receptors in depression: Pinnock R. D., et al., Brain Res., 1994;653, 199.

Psychoses

The compounds of the invention are useful in the treatment of psychoses. The following publication provides evidences of the role of bombesin receptors in psychoses: Merali., et al., Eur. J. Pharmacol., 1990;191, 281.

Sleeping Disorders

The compounds of the invention are useful in the treatment of sleep disorders. The following publication provides evidences of the role of bombesin receptors in sleeping disorders: Even PC., et al., Physiol behav., 1991; 49(3):439-42.

Memory Impairment

The compounds of the invention are useful in the treatment of memory impairment. The following publication provides evidences of the role of bombesin receptors in memory impairment: Rashidy., et al., Brain Research., 1998; 814:127-32.

Pulmonary Hypertension

Hurel S. J. et al. (Lancet (1996) 348: 1243) have shown that infusion of a GRP receptor antagonist to a patient suffering from pulmonary hypertension was followed by a decrease in the pulmonary systolic pressure. The compounds of the invention are useful in the treatment of pulmonary hypertension.

Lung Repair and Lung Development Disorders

Several studies have emphasised the role of GRP and the GRP receptor in lung repair after injury and in lung development (Spurzem J. R. et al. (1997) Am. J. Respir. Cell. Mol. Biol. 16: 209-211; Wang D. . et al. (1996) Am. J. Respir. Cell. Mol. Biol. 14: 409-416; Spindel E. R., Ibidem 14: 407-408). Also, lung injury, including that induced by smoking, leads to increased levels of pulmonary bombesin-like peptides. Findings by Cutz E. et al. (Pediatrics (1996) 98: 668-72) suggest that maternal smoking potentiates hyperplasia of the pulmonary neuroendocrine cells (as measured by the percentage of airway epithelium immunoreactive for bombesin) in the lungs of infants who die of sudden infant death syndrome (SIDS) and that a dysfunction of these cells may contribute to the pathophysiology of SIDS. The compounds of the instant invention are useful in the treatment of lung repair and lung development disorders.

Cancer Treatment

The invention also relates to a method for treating cancer which comprises administering to a patient or a subject, particularly a mammal, more particularly a human, an effective amount of a compound of Formula (I), optionally conjugated with a cytotoxic agent. The method is particularly useful in cancers where tumour cells have a cell surface bombesin receptor, including certain prostate or pancreatic cancers.

When a directly labelled compound of Formula (I) is used for therapeutic purposes, preferably a halogen substituent of Ar as a radionuclide is used. Preferably halogen radionuclides employed for therapy are β-emifting or α-emitting radio-nuclides. The preferred halogen substituents of Ar for treating cancers include ¹³¹¹I, ²¹¹At, ⁷⁶Br and ⁷⁷Br, ¹³¹I being particularly preferred. Compounds of Formula (I) where Ar is substituted by a radionuclide halogen can easily be prepared via electrophilic aromatic substitution of a corresponding non-radioactive compound wherein Ar is substituted by a halide or an activating group. Such a halide is preferably Br or I. Preferred activating groups include tributyl-tin, trimethylsilyl, t-butyldimethylsilyl, and the like.

Conjugation of a compound of Formula (I) with a cytotoxic agent is especially preferred when, in the compound of Formula (I), R² is hydroxy or amino. In such a case, the compounds of the invention may conveniently be linked to a cytotoxic agent, using a bifunctional moiety like glutaric acid or the like to form a conjugate. Suitable cytotoxic agents include compounds such as doxorubicin, anticancer chemotherapy compounds such as those described in The Merck Index, 12th edition, 1996, p. MISC-10.

The use of a conjugate of a compound of Formula (I) with a radionuclide is also provided by the instant invention; preferred radionuclides used for radiotherapy emit an a or P particle; they include ¹⁸⁸Re, ¹³¹I, ²¹¹At, ²¹²Pb ²¹²Bi, ⁷⁶Br, ⁷⁷Br, and the like (for examples, The Merck Index, 12th edition, 1996, page MISC-93). Said conjugates may be prepared using conventional methods. For example, radionuclides such as ¹⁸⁸Re can be linked to a compound of Formula (I) using a bifunctional chelating agent such as trisuccin (Safavy A. et al. (1993) Bioconj. Chem. 4: 194-8) according to a process adapted from Safavy A. et al. in Cancer (1997) 80 (Suppl): 2354-9. The conjugate may take the form of a compound that is cleaved to release the cytotoxic agent on entry into the tumour cells. Compounds that are rapidly transformed in vivo to yield the parent compound of the above formulae, e.g. by hydrolysis upon entry into a target cell, are preferred.

A method of the present invention for treating a mammalian tumour includes administering to a mammal a composition including a tumour-inhibiting amount of at least one compound of the present invention. Such a tumour-inhibiting amount is an amount of at least one of the subject compounds which permits sufficient tumour localisation of the compound to diminish tumour growth or size. This dosage can range from about 0.1 mmol/kg body weight to about 500 mmol/kg body weight. A preferred dosage is about 5 to about 50 mmol/kg body weight.

The amount of radioactivity administered can vary depending on the type of radionuclide. However, with this in mind the amount of radioactivity that is administered can vary from about 1 millicurie (mCi) to about 800 mCi. Preferably, about 10 mCi to about 600 mCi is administered. Moreover when considering the dosage, the specific activity of the radioactive compound should be taken into consideration. Such a specific activity is preferably very high, e.g. for ¹²³I-labelled compounds the specific activity should be at least about 1,000 Ci/mM to about 50,000 Ci/mM. More preferably the specific activity for ¹²³I-labelled compounds is, e.g., about 10,000 Ci/mM to about 22,000 Ci/mM.

a) Prostate Cancer

Bombesin specifically induces intracellular calcium mobilisation via GRP receptors in human prostate cancer cells (Aprikian A. G. et al.(1996) J. Mol. Endocrinol 16: 297-306). This suggests that the bombesin family of neuropeptides can play a regulatory role in the biology of prostate cancer. The use of antibodies raised against bombesin inhibited the growth of a prostatic carcinoma cell line (Hoosein N. M., (1993) Cancer Bull. 45:436-441).

The compounds of the instant invention are useful in the diagnosis and treatment of prostate cancer.

b) Pancreatic Cancer

Normal and tumour pancreatic cells contain a specific GRP receptor that is expressed more on malignant pancreatic tissues (Hajri A. et al.(1996) Pancreas 12: 25-35). Bombesin-like peptides may stimulate proliferation of human pancreatic cancer cells (Wang Q. J. et al. Int. J. Cancer (1996) 68: 528-34). As a consequence a bombesin receptor antagonist may be used to treat pancreatic cancers. Furthermore, a radiolabelled bombesin receptor antagonist may be used to treat pancreatic cancers.

The compounds of the instant invention are useful in the treatment of pancreatic cancer.

Hepatic Porphyria

The major clinical manifestation of hepatic porphyrias are neurologic symptoms, including abdominal pain, neuropathy, and mental disturbances. It is believed that the neurologic symptoms are caused by an increase of a few gastrointestinal and neurotransmitter polypeptides, including GRP, in the systemic circulation during the acute phase of the disease (Medenica R. et al. (1997) Cell Mol. Biol. 43: 9-27). Treatment with bombesin receptor antagonists may thus reduce the effects of those polypeptides that bind to bombesin receptors, and alleviate the symptomatology of acute porphyria. The compounds of the instant invention are useful in the treatment of hepatic porphyria.

Gastrointestinal Secretory Disturbances

GRP has proved to be a particularly valuable tool in detecting disturbances of gastric secretory function, including those associated with duodenal ulcer disease and Helicobacter pylori infection (McColl K. E. et al. (1995) Aliment. Pharmacol. Ther. 9: 341-7). As a consequence, a radiolabelled bombesin receptor antagonist may be useful to diagnose these conditions. Other gastrointestinal functions such as gallbladder contraction, pancreatic secretion and gastro-oesophageal motility are subject to regulatory controls by GRP, and a radiolabelled bombesin receptor antagonist may be useful to diagnose these conditions.

The compounds of the instant invention are useful in the treatment of gastrointestinal secretory disturbances.

Gastrointestinal Disorders

The bombesin receptor has been implicated in gastric acid secretion and gastrointestinal motility Walsh J. H. Ann. Rev Physiol 1988; 50, 41 and Lebacq-Verheyden A et al., in Handbook of Experimental pharmacology 1990;95 (part II) and references therein). As such it could be implicated in colitis, Crohn's disease and inflammatory bowel disease.

Emesis

Bombesin is present in high concentrations in the skin of frogs. As part of a defence reaction, Amphibia secrete emetic substances when swallowed by a predator.

In mammals, bombesin receptors are widely distributed in the GI tract where they cause changes in gastric motility and secretion. Bombesin receptor antagonists of the invention may decrease retching and vomiting and thus be effective in the treatment of emesis, in particular in patients receiving anticancer agents.

Anorexia

Bombesin causes a decrease of glucose intake in mice. In mice lacking the GRP receptor, bombesin no longer showed this effect (Hampton L. et al, Proc. Natl. Acad. Sci. USA, 95: 3188-92, 1998). Bombesin receptor antagonists used in the present invention may increase feeding behavior, and thus be effective in the treatment of anorexia, such as the anorexia of cancer patients.

Pain

The compounds of the invention are useful in the treatment of pain. The following publication provides evidences of the role of bombesin receptors in pain (Cridland and Henry, Brain Research, 584: 163-168, 1992).

Seasonal Affective Disorders

The compounds of the invention are useful in the treatment of seasonal affective disorders. The following publication provides evidences of the role of bombesin receptors in seasonal affective disorders: McArthur A J., et al., J. Neurosci., 2000; 20(14):5496-502.

Feeding Disorders

The compounds of the invention are useful in the treatment of feeding disorders. The following publication provides evidences of the role of bombesin receptors in feeding disorders: Ladenheim E E., et al, 1996, 54:705-711.

Pruritus

The compounds of the invention are useful in the treatment of pruritus. The following publication provides evidences of the role of bombesin receptors in pruritus: Maigret C. et al, Eur. J. Pharmacol., 209: 57-61, 1991.

Protocol for BB₁ and BB₂ Binding Assays

In the following experiments, measurement of BB₁ and BB₂ binding was as follows. CHO-K1 cells stably expressing cloned human NMB (for (BB₁ assay) and GRP receptors (for BB₂ assay) were routinely grown in Ham's F12 culture medium supplemented with 10% foetal calf serum and 2 mM glutamine. For binding experiments, cells were harvested by trypsinization, and stored frozen at −70° C. in Ham's F12 culture medium containing 5% DMSO until required. On the day of use, cells were thawed rapidly, diluted with an excess of culture medium, and centrifuged for 5 minutes at 2000 g. Cells were resuspended in 50 mM Tris-HCl assay buffer (pH 7.4 at 21° C., containing 0.02% BSA, 40 mg/mL bacitracin, 2 mg/mL chymostatin, 4 mg/mL leupeptin, and 2 mM phosphoramidon), counted, and polytronned (setting 5, 10 sec) before centrifuging for 10 minutes at 28,000 g. The final pellet was resuspended in assay buffer to a final cell concentration of 1.5×10⁵/mL. For binding assays, 200 μL aliquots of membranes were incubated with [¹²⁵I][Tyr⁴]bombesin (<0.1 nM) in the presence and absence of test compounds (final assay volume 250 μL) for 60 minutes and 90 minutes for NMB and GRP receptors, respectively. Nonspecific binding was defined by 1 μM bombesin. Assays were terminated by rapid filtration under vacuum onto Whatman GF/C filters presoaked in 0.2% PEI for >2 hours, and washed 50 mM Tris-HCl (pH 6.9 at 21° C.; 6×1 mL). Radioactivity bound was determined using a gamma counter.

All competition data was analysed using nonlinear regression utilising iterative curve-plolting procedures in Prism® (GraphPad Software Inc., San Diego, USA). IC₅₀ values were corrected to Kᵢ values using the Cheng-Prusoff equation (Cheng Y., Prusoff W. H., Biochem. Pharmacol. 22: 3099-3108, 1973).

Preparative Methods

Throughout this application the following abbreviation have the meanings listed below:

NEt₃ triethylamine

THF tetrahydrofuran

HBTU O-benzotriazol-1-yl-N,N,N′,N′-Atetramethyluronium hexafluorophosphate

DIPEA N,N-diisopropylethylamine

DMF N,N-dimethylformamide

TEBA benzyltriethylammonium chloride

BOC₂O di-tert-butyl dicarbonate

TFA trifluoroacetic acid

DMA N,N-dimethylacetamide

EtOAc EtOAc

MeOH methanol

Trp tryptophan

Ph phenyl

HPLC high pressure liquid chromatography

NP normal phase

RP reverse phase

MAP N,N-dimethyl-4-aminopyridine

OAc acetate

OB oestradiol benzoate.

How the invention may be put into effect will now be further described with reference to the following examples.

### SYNTHESIS EXAMPLE

### (S)-2-Amino-3-(1H-indol-3-yl)-2-methyl-N-(1-pyridin-2-yl-cyclohexylmethyl)-propionamide (Intermediate VIa) and

### (S)-2-Amino-3-(1H-indol-3-yl)-2-methyl-N-(1-(5-methoxy-pyridin-2-yl)-cyclohexylmethyl)-propionamide (Intermediate VIb).

In reaction scheme 1 below, Intermediates VIa and VIb are made by (i) 30 protecting the amino group of the starting amino acid 1 with di-t-butyl carbonate (BOC₂O) and potassium carbonate in dioxane/water, (ii) forming an amide by reaction of the N-protected amino acid with an amine 2a or 2b in dimethylformamide in the presence of O-benzotriazol-1-yl-N,N,N′,N′-tetramethyluronium hexafluorophosphate (HBTU) and N,N-diisopropyl-ethylamine (DIPEA), and (iii) deprotecting the amino group of the product 3a or 3b by reaction with trifluoroacetic acid (TFA) in dichloromethane.

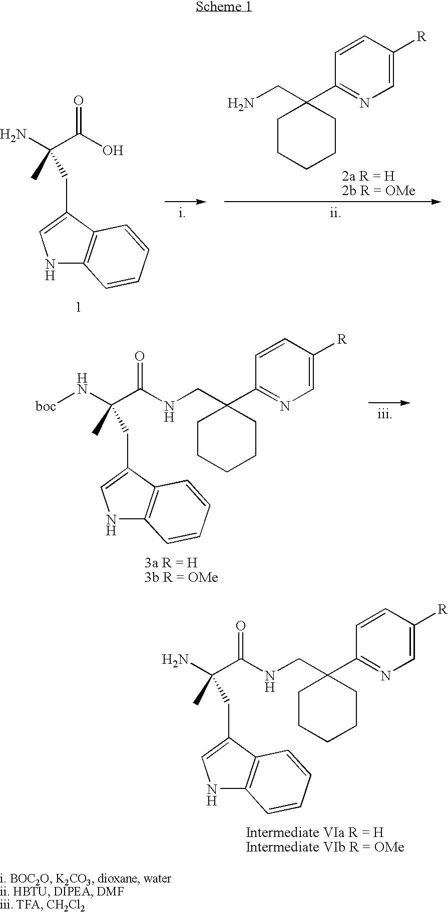

### {(S)-2-(1-H-Indol-3-yl)-1-methyl-1-[(1-pyridin-2-yl-cyclohexylmethyl)-carbamoyl]-ethyl}-carbamic acid tert-butyl ester (3a)

(1) To a stirred solution of H-(S)-αMeTrp-OH (1) (10 g, 46 mmol) and di-t-butyl-dicarbonate (10 g, 46 mmol) in dioxane (100 ml) was added water (20 ml) and potassium carbonate (10 g, 74 mmol). After 4 hours the reaction mixture was acidified with 2N hydrochloric acid (150 ml) and product extracted with EtOAc (2×200 ml). The combined organic phases were dried (MgSO₄) and evaporated under reduced pressure. The residue was purified by flash chromatography, eluting with EtOAc. Removal of solvent under reduced pressure gave Boc-(S)-αMeTrp-OH as orange oil (14.5 g, 99%).

(2) To a stirred solution of Boc-(S)-αMeTrp-OH (7 g, 22 mmol) in DMF (100 ml) was added HBTU (8.0 g, 22 mmol), NEt₃ (5 ml, 35 mmol), and [1-(2-pyridyl)cyclohexyl]methylamine (2, 4.2 g, 22 mmol, described in WO 9807718). After 1 hour the reaction mixture was diluted with EtOAc (300 ml) and washed with 2N hydrochloric acid (2×200 ml), dried (MgSO₄) and evaporated under reduced pressure at 60° C. The residue was purified by flash chromatography. Elution with 5% methanol in dichloromethane and subsequent removal of solvent under reduced pressure gave 3a as yellow oil (8.3 g, 77%).

IR (film): 3339, 2929, 2858, 1704, 1659, 1651, 1589, 1519, 1487, 1366, 1249, 1164, 1070, 908, 737 cm⁻¹;

NMR (CDCl₃): δ=1.20-1.70 (20H, m), 2.00-2.12 (2H, m), 3.25-3.50 (4H, m), 5.05-5.20 (1H, br.s), 6.92 (1H, d, J=2.0 Hz), 7.02-7.32 (6H, m), 7.51 (1H, d, J=8.0 Hz), 7.59-7.64 (1H, m), 8.03 (1H, s), 8.48 (1H, d, J=4 Hz);

MS m/e (AP+): 491 (M⁺+H, 100%), 513 (M⁺+Na, 20%).

### (3) (S)-2-Amino-3-(1H-indol-3-yl)-2-methyl-N-(1-pyridin-2-yl-cyclohexyl methyl)-propionamide (Intermediate VIa)

To a stirred solution of 3a (8.2 g, 16.5 mmol) in dichloromethane (100 ml) was added trifluoroacetic acid (3.0 ml, 39 mmol). After 18 hours the solvent was removed under reduced pressure at 60° C. The residue was treated cautiously with saturated sodium carbonate solution (200 ml) before extracting with EtOAc (3×200 ml). The combined organic phases were dried (MgSO₄) and evaporated under reduced pressure at 60° C. The residue was purified by flash chromatography. Elution with 0-5% methanol in dichloromethane and subsequent removal of solvent under reduced pressure gave Intermediate Via as white foam (4.85 g, 75%).

MPt: 65-68° C.;

IR (KBr disc): 3367, 2926, 2855,1648, 1589, 1569, 1522,1455, 1430, 1366, 1341, 1234, 842, 784, 742 cm⁻¹;

NMR (CbCl₃): δ=1.20-1.80 (13H, m), 1.98-2.20 (2H, m), 2.83 (1H, d, J=14.2 Hz), 3.33 (1H, d, J=14.2 Hz), 3.38 (2H, d, J=5.6 Hz), 6.98-7.20 (6H, m), 7.50-7.75 (3H, m), 8.05-8.15 (1H, s), 8.49-8.51 (1H, m);

MS m/e (AP+): 391 (M⁺+H, 100%).

### {(S)-2-(1-H-Indol-3-yl)-1-methyl-1-[(1-(5-methoxy-pyridin-2-yl)-cyclohexylmethyl)-carbamoyl]-ethyl}-carbamic acid tert-butyl ester (3b)

To a stirred solution of Boc-(S)-□MeTrp-OH (1.44 g, 4.5 mmol) in DMF (50 ml) was added HBTU (1.72 g, 4.5 mmol), DIPEA (2.38 ml, 13.6 mmol), and [1-(5-methoxy-2-pyridyl)cyclohexyl]methanamine (1 g, 4.5 mmol). After over night the reaction mixture was diluted with EtOAc (300 ml) and water, dried (MgSO₄) and evaporated under reduced pressure. The residue was purified by flash chromatography. Elution with EtOAc/heptane (1:1) and subsequent removal of solvent under reduced pressure gave 3b as an oil (2.207 g, 94%).

NMR (CDCl₃): □=1.24-1.60 (8H, m), 1.39 (9H, s), 1.52 (3H, s), 2.00-2.18 (2H, m), 3.20-3.43 (4H, m), 3.82 (3H, s), 6.92 (1H, d, J=2.4 Hz), 7.02-7.20 (6H, m), 7.30 (1H, d, J=6.0 Hz), 7.51 (1H, d, J=8 Hz), 8.00 (1H, s), 8.17 (1H, d, J=2.8 Hz).

MS m/e (ES+): 521.36 (M⁺+H, 100%), 543.25 (M⁺+Na).

Intermediate VIb

To a stirred solution of 3b (2.2 g, 4.2 mmol) in dichloromethane (10 ml) was added trifluoroacetic acid (5 ml, excess). After stirring over night the reaction mixture was taken up in 1 N HCl and extracted with diethylether. Organic phase discarded. The aqueous phase was basified cautiously with saturated sodium carbonate solution before extracting with EtOAc (3×50 ml). The combined organic phases were dried (MgSO₄) and evaporated under reduced pressure at 60° C. to give Intermediate VIb as a glass (1.253 g, 71%).

IR (film): 3272, 2930, 2857, 1651, 1595, 1573, 1520, 1489, 1478, 1455, 1393, 1358, 1291, 1268, 1232, 1181, 1150, 1131, 1030, 1012, 831, 741 cm⁻¹;

NMR (DMSO): ▭=1.10-1.65 (13H, m), 1.80-1.90 (1H, m), 2.00-2.10 (1H, m), 2.70 (1H, d, J=13.9 Hz), 3.10 (1H, d, J=13.9 Hz), 3.10-3.22 (2H, m), 3.77 (3H,s), 6.93-7.07 (4H, m), 7.16-7.19 (1H, m), 7.32 (1H, d, J=8.1 Hz), 7.48-7.55 (2H, m), 8.21 (1H, d, J=3.2 Hz), 10.88 (1H, s);

MS m/e (ES+): 421.27 (M⁺+H, 100%), 443.26 (M⁺+Na).

### Examples 1-55

### N-acyl Derivatives of Intermediate VIa and VIb

Scheme 2 describes the synthesis of N-acyl derivatives of Intermediates VIa and VIb.

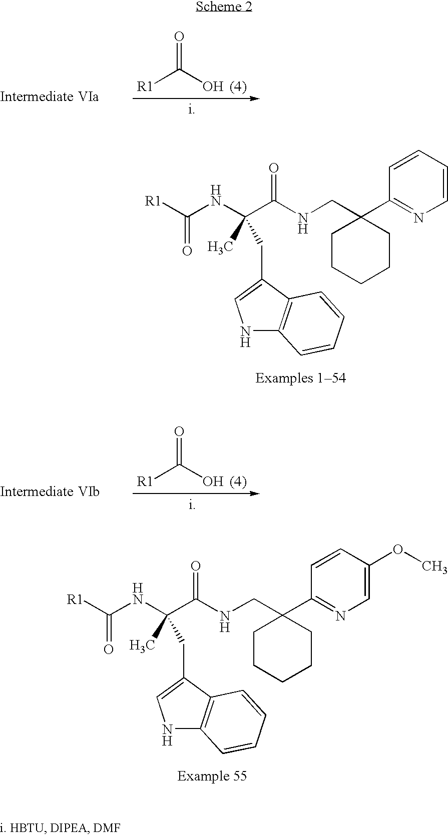

In scheme 2, R1 represents the rest of the carboxylic acid (4) molecule. These intermediates (4) are listed in table 1

N-acyl Derivatives of Intermediate VIa

To acid 4 (0.18 mmol) was added 0.50 M HBTU in DMF (300 μL, 0.15 mmol), 1.0 M diisopropylethylamine in DMF (300 μL, 0.30 mmol) and 0.40 M Intermediate VIa in DMF (375 μL, 0.15 mmol). The solution was shaken on an orbital shaker at room temperature for 18 h. Water (1.0 mL) was added and the mixture was loaded onto a LC-18 SPE cartridge (0.5 g sorbent) and the cartridge was eluted with water (3 mL), 25% methanol/water (3 mL), 50% methanol/water (4 mL) and methanol (4.5 mL)). The methanol fraction was concentrated and analysed by LCMS. When the purity was <90% the product was further purified by prep. HPLC (column: Phenomenex primesphere 10μ C18-HC 110A, 100×21.20 mm; mobile phase: methanol/water 10 to 100% gradient). The products were characterised and analysed by LCMS (column: 50×4.6 mm Prodigy ODSIII (5μ) column; mobile phase: acetonitrile/water (0.1% formic acid) 5 to 100% gradient over 2 min, held at 100% acetonitrile for 1 min; flow rate 4 mL/min; UV detection at 215 nm; mass spec: 150-900 Da full scan APCI+ centroid data)

The following products were made by the above method, with the starting material listed in Table 1 and gave the test results indicated in Table 2:

TABLE 1

| Example | Intermediate 4 |
|---|---|
| 1 | Benzoic acid |
| 2 | 4-Methyl-benzoic acid |
| 3 | 4-Chloro-benzoic acid |
| 4 | 4-Methoxy-benzoic acid |
| 5 | 4-Nitro-benzoic acid |
| 6 | 4-Methanesulfonyl-benzoic acid |
| 7 | 3-Cyano-benzoic acid |
| 8 | 3-Chloro-benzoic acid |
| 9 | 3-Methoxy-benzoic acid |
| 10 | 3-Methanesulfonyl-benzoic acid |
| 11 | 3-Dimethylamino-benzoic acid |
| 12 | 3-Methyl-benzoic acid |
| 13 | 2-Chloro-benzoic acid |
| 14 | 2-Nitro-benzoic acid |
| 15 | 2-Methoxy-benzoic acid |
| 16 | 2-Methyl-benzoic acid |
| 17 | 2-Dimethylamino-benzoic acid |
| 18 | 2-Fluoro-benzoic acid |
| 19 | p-Tolyl-acetic acid |
| 20 | o-Tolyl-acetic acid |
| 21 | (4-Hydroxy-phenyl)-acetic acid |
| 22 | (3-Hydroxy-phenyl)-acetic acid |
| 23 | m-Tolyl-acetic acid |
| 24 | (2-Fluoro-phenyl)-acetic acid |
| 25 | Thiophen-3-yl-acetic acid |
| 26 | Pyridine-2-carboxylic acid |
| 27 | Isonicotinic acid |
| 28 | Furan-3-carboxylic acid |
| 29 | Furan-2-carboxylic acid |
| 30 | 1H-Indole-2-carboxylic acid |
| 31 | 5-Methyl-isoxazole-3-carboxylic acid |
| 32 | 1-Methyl-1H-pyrrole-2-carboxylic acid |
| 33 | Thiophene-2-carboxylic acid |
| 34 | Thiophene-3-carboxylic acid |
| 35 | 1H-Indole-6-carboxylic acid |
| 36 | 1H-Indole-5-carboxylic acid |
| 37 | 1H-Indole-4-carboxylic acid |
| 38 | 1H-Indole-7-carboxylic acid |
| 39 | 1-Methyl-1H-indole-2-carboxylic acid |
| 40 | Benzo[b]thiophene-2-carboxylic acid |
| 41 | Benzothiazole-6-carboxylic acid |
| 42 | 1H-Benzotriazole-5-carboxylic acid |
| 43 | 3-Methyl-thiophene-2-carboxylic acid |
| 44 | 5-Methyl-thiophene-2-carboxylic acid |
| 45 | 6-Methyl-pyridine-2-carboxylic acid |
| 46 | Isoquinoline-3-carboxylic acid |
| 47 | Quinoxaline-2-carboxylic acid |
| 48 | Quinoline-8-carboxylic acid |
| 49 | 5-Phenyl-oxazole-4-carboxylic acid |
| 50 | 2-Pyrrol-1-yl-benzoic acid |
| 51 | (4-Methoxy-phenyl)-acetic acid |
| 52 | (4-Dimethylamino-phenyl)-acetic acid |
| 53 | (2-Nitro-phenyl)-acetic acid |
| 54 | (2-Methoxy-phenyl)-acetic acid |
| 55 | 1H-Indole-2-carboxylic acid |

TABLE 2

| | | | | LCMS | | |
|---|---|---|---|---|---|---|
| | | | | Ret | BB₁ | BB₂ |
| Example | | | Purity | time | IC50 | IC50 |
| No | Product | MH⁺ | % | (min) | (nM) | (nM) |
| 1 | N {(S)-2-(1H-Indol-3-yl)-1-methyl-1- | 494, 64 | 100 | 1.71 | 2499 | IA |
| | [(1-pyridin-2-yl-cyclohexylmethyl)- | | | | | |
| | carbamoyl]-ethyl}-benzamide | | | | | |
| 2 | N {(S)-2-(1H-Indol-3-yl)-1-methyl-1- | 508, 67 | 95 | 1.76 | 2499 | IA |
| | [(1-pyridin-2-yl-cyclohexylmethyl)- | | | | | |
| | carbamoyl]-ethyl}-4-methyl- | | | | | |
| | benzamide | | | | | |
| 3 | 4-Chloro-N {(S)-2-(1H-indol-3-yl)-1- | 529, 09 | 94 | 1.84 | 1349 | IA |
| | methyl-1-[(1-pyridin-2-yl-cyclohexyl- | | | | | |
| | methyl)-carbamoyl]-ethyl}- | | | | | |
| | benzamide | | | | | |
| 4 | N {(S)-2-(1H-Indol-3-yl)-1-methyl-1- | 524, 67 | 94 | 1.68 | 2879 | IA |
| | [(1-pyridin-2-yl-cyclohexylmethyl)- | | | | | |
| | carbamoyl]-ethyl}-4-methoxy- | | | | | |
| | benzamide | | | | | |
| 5 | N {(S)-2-(1H-Indol-3-yl)-1-methyl-1- | 539, 64 | 80 | 1.79 | 343 | IA |
| | [(1-pyridin-2-yl-cyclohexylmethyl)- | | | | | |
| | carbamoyl]-ethyl}-4-nitro-benzamide | | | | | |
| 6 | N {(S)-2-(1H-Indol-3-yl)-1-methyl-1- | 572, 73 | 95 | 1.60 | 2272 | IA |
| | [(1-pyridin-2-yl-cyclohexylmethyl)- | | | | | |
| | carbamoyl]-ethyl}-4- | | | | | |
| | methanesulfonyl-benzamide | | | | | |
| 7 | 3-Cyano-N {(S)-2-(1H-indol-3-yl)-1- | 519, 65 | 91 | 1.71 | 2042 | IA |
| | methyl-1-[(1-pyridin-2-yl- | | | | | |
| | cyclohexylmethyl)-carbamoyl]-ethyl}- | | | | | |
| | benzamide | | | | | |
| 8 | 3-Chloro-N {(S)-2-(1H-indol-3-yl)-1- | 529, 09 | 97 | 1.84 | 1269 | IA |
| | methyl-1-[(1-pyridin-2-yl-cyclohexyl- | | | | | |
| | methyl)-carbamoyl]-ethyl}- | | | | | |
| | benzamide | | | | | |
| 9 | N {(S)-2-(1H-Indol-3-yl)-1-methyl-1- | 524, 67 | 98 | 1.73 | 2859 | IA |
| | [(1-pyridin-2-yl-cyclohexylmethyl)- | | | | | |
| | carbamoyl]-ethyl}-3-methoxy- | | | | | |
| | benzamide | | | | | |
| 10 | N {(S)-2-(1H-Indol-3-yl)-1-methyl-1- | 572, 73 | 95 | 1.60 | 3051 | IA |
| | [(1-pyridin-2-yl-cyclohexylmethyl)- | | | | | |
| | carbamoyl]-ethyl}-3- | | | | | |
| | methanesulfonyl-benzamide | | | | | |
| 11. | Dimethylamino-N {(S)-2-(1H-indol-3- | 537, 71 | 91 | 1.74 | 2518 | IA |
| | yl)-1-methyl-1-[(1-pyridin-2-yl- | | | | | |
| | cyclohexyl-methyl)-carbamoyl]- | | | | | |
| | ethyl}-benzamide | | | | | |
| 12 | N {(S)-2-(1H-Indol-3-yl)-1-methyl-1- | 508, 67 | 100 | 1.79 | 2351 | IA |
| | [(1-pyridin-2-yl-cyclohexylmethyl)- | | | | | |
| | carbamoyl]-ethyl}-3-methyl- | | | | | |
| | benzamide | | | | | |
| 13 | 2-Chloro-N {(S)-2-(1H-indol-3-yl)-1- | 529, 09 | 98 | 1.79 | 3229 | IA |
| | methyl-1-[(1-pyridin-2-yl-cyclohexyl- | | | | | |
| | methyl)-carbamoyl]-ethyl}- | | | | | |
| | benzamide | | | | | |
| 14 | N {(S)-2-(1H-Indol-3-yl)-1-methyl-1- | 539, 64 | 91 | 1.71 | 4581 | IA |
| | [(1-pyridin-2-yl-cyclohexylmethyl)- | | | | | |
| | carbamoyl]-ethyl}-2-nitro-benzamide | | | | | |
| 15 | N {(S)-2-(1H-Indol-3-yl)-1-methyl-1- | 524, 67 | 100 | 1.73 | 2559 | IA |
| | [(1-pyridin-2-yl-cyclohexylmethyl)- | | | | | |
| | carbamoyl]-ethyl}-2-methoxy- | | | | | |
| | benzamide | | | | | |
| 16 | N {(S)-2-(1H-Indol-3-yl)-1-methyl-1- | 508, 67 | 100 | 1.79 | 3283 | IA |
| | [(1-pyridin-2-yl-cyclohexylmethyl)- | | | | | |
| | carbamoyl]-ethyl}-2-methyl- | | | | | |
| | benzamide | | | | | |
| 17 | C-Dimethylamino-N {(S)-2-(1H-indol- | 537, 71 | 93 | 1.79 | 716 | IA |
| | 3-yl)-1-methyl-1-[(1-pyridin-2-yl- | | | | | |
| | cyclohexyl-methyl)-carbamoyl]- | | | | | |
| | ethyl}-benzamide | | | | | |
| 18 | 2-Fluoro-N {(S)-2-(1H-indol-3-yl)-1- | 512, 63 | 98 | 1.76 | 3949 | IA |
| | methyl-1-[(1-pyridin-2-yl- | | | | | |
| | cyclohexylmethyl)-carbamoyl]-ethyl}- | | | | | |
| | benzamide | | | | | |
| 19 | (S)-3-(1H-Indol-3-yl)-2-methyl-N-(1- | 522, 70 | 94 | 1.76 | 944 | IA |
| | pyridin-2-yl-cyclohexylmethyl)-2-(2-p- | | | | | |
| | tolyl-ethanoylamino)-propionamide | | | | | |
| 20 | (S)-3-(1H-Indol-3-yl)-2-methyl-N-(1- | 522, 70 | 98 | 1.76 | 944 | IA |
| | pyridin-2-yl-cyclohexylmethyl)-2-(2-o- | | | | | |
| | tolyl-ethanoylamino)-propionamide | | | | | |
| 21 | (S)-2-[2-(4-Hydroxy-phenyl)- | 524, 67 | 96 | 1.50 | 3135 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 22 | (S)-2-[2-(3-Hydroxy-phenyl)- | 524, 67 | 90 | 1.52 | 1437 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 23 | (S)-3-(1H-Indol-3-yl)-2-methyl-N-(1- | 522, 70 | 95 | 1.76 | 817 | IA |
| | pyridin-2-yl-cyclohexylmethyl)-2-(2- | | | | | |
| | m-tolyl-ethanoylamino)- | | | | | |
| | propionamide | | | | | |
| 24 | (S)-2-[2-(2-Fluoro-phenyl)- | 526, 66 | 94 | 1.71 | 878 | 1546 |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 25 | (S)-3-(1H-Indol-3-yl)-2-methyl-N-(1- | 514, 70 | 93 | 1.65 | 1437 | IA |
| | pyridin-2-yl-cyclohexylmethyl)-2-(2- | | | | | |
| | thiophen-3-yl-ethanoylamino)- | | | | | |
| | propionamide | | | | | |
| 26 | Pyridine-2-carboxylic acid {(S)-2- | 495, 63 | 98 | 1.68 | 3709 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 27 | N {(S)-2-(1H-Indol-3-yl)-1-methyl-1- | 495, 63 | 98 | 1.47 | 1365 | IA |
| | [(1-pyridin-2-yl-cyclohexylmethyl)- | | | | | |
| | carbamoyl]-ethyl}-isonicotinamide | | | | | |
| 28 | Furan-3-carboxylic acid {(S)-2-(1H- | 484, 60 | 97 | 1.60 | 1204 | IA |
| | indol-3-yl)-1-methyl-1-[(1-pyridin-2- | | | | | |
| | yl-cyclohexylmethyl)-carbamoyl]- | | | | | |
| | ethyl}-amide | | | | | |
| 29 | Furan-2-carboxylic acid {(S)-2-(1H- | 484, 60 | 100 | 1.60 | 1204 | IA |
| | indol-3-yl)-1-methyl-1-[(1-pyridin-2- | | | | | |
| | yl-cyclohexylmethyl)-carbamoyl]- | | | | | |
| | ethyl}-amide | | | | | |
| 30 | 1H-Indole-2-carboxylic acid {(S)-2- | 533, 68 | 100 | 1.79 | 289 | 527 |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 31 | 5-Methyl-isoxazole-3-carboxylic acid | 499, 62 | 94 | 1.46 | 4127 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 32 | 1-Methyl-1H-pyrrole-2-carboxylic | 497, 65 | 96 | 1.46 | 4819 | — |
| | acid {(S)-2-(1H-indol-3-yl)-1-methyl- | | | | | |
| | 1-[(1-pyridin-2-yl-cyclohexylmethyl)- | | | | | |
| | carbamoyl]-ethyl}-amide | | | | | |
| 33 | Thiophene-2-carboxylic acid {(S)-2- | 500, 67 | 100 | 1.42 | 1437 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 34 | Thiophene-3-carboxylic acid {(S)-2- | 500, 67 | 100 | 1.39 | 2201 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 35 | 1H-Indole-6-carboxylic acid {(S)-2- | 533, 68 | 100 | 1.42 | 1604 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 36 | 1H-Indole-5-carboxylic acid {(S)-2- | 533, 68 | 100 | 1.35 | 1881 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 37 | 1H-Indole-4-carboxylic acid {(S)-2- | 533, 68 | 99 | 1.35 | 4503 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 38 | 1H-Indole-7-carboxylic acid {(S)-2- | 533, 68 | 100 | 1.60 | 1369 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 39 | 1-Methyl-1H-indole-2-carboxylic acid | 547, 71 | 100 | 1.70 | 1233 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 40 | Benzo[b]thiophene-2-carboxylic acid | 550, 73 | 100 | 1.63 | 611 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 41 | Benzothiazole-6-carboxylic acid {(S)- | 551, 72 | 95 | 1.35 | 897 | 1495 |
| | 2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 42 | 1H-Benzotriazole-5-carboxylic acid | 535, 65 | 95 | 1.25 | 3167 | — |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 43 | 3-Methyl-thiophene-2-carboxylic acid | 514, 70 | 100 | 1.53 | 744 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 44 | 5-Methyl-thiophene-2-carboxylic acid | 514, 70 | 100 | 1.60 | 1663 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 45 | 6-Methyl-pyridine-2-carboxylic acid | 509, 66 | 98 | 1.6 | 2816 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 46 | Isoquinoline-3-carboxylic acid {(S)-2- | 545, 69 | 100 | 1.71 | 1363 | — |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 47 | Quinoxaline-2-carboxylic acid {(S)-2- | 546, 68 | 94 | 1.67 | 1425 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 48 | Quinoline-8-carboxylic acid {(S)-2- | 545, 69 | 96 | 1.57 | 4479 | IA |
| | (1H-indol-3-yl)-1-methyl-1-[(1- | | | | | |
| | pyridin-2-yl-cyclohexylmethyl)- | | | | | |
| | carbamoyl]-ethyl}-amide | | | | | |
| 49 | 5-Phenyl-oxazole-4-carboxylic acid | 561, 69 | 95 | 1.81 | 2660 | IA |
| | {(S)-2-(1H-indol-3-yl)-1-methyl-1-[(1- | | | | | |
| | pyridin-2-yl-cyclohexylmethyl)- | | | | | |
| | carbamoyl]-ethyl}-amide | | | | | |
| 50 | N {(S)-2-(1H-Indol-3-yl)-1-methyl-1- | 559, 72 | 98 | 1.71 | 361 | IA |
| | [(1-pyridin-2-yl-cyclohexylmethyl)- | | | | | |
| | carbamoyl]-ethyl}-2-pyrrol-1-yl- | | | | | |
| | benzamide | | | | | |
| 51 | (S)-3-(1H-Indol-3-yl)-2-[2-(4- | 538, 70 | 98 | 1.71 | 1694 | IA |
| | methoxy-phenyl)-ethanoylamino]-2- | | | | | |
| | methyl-N-(1-pyridin-2-yl- | | | | | |
| | cyclohexylmethyl)-propionamide | | | | | |
| 52 | (S)-2-[2-(4-Dimethylamino-phenyl)- | 551, 74 | 100 | 1.36 | 2708 | IA |
| | ethanoylamino]-3-(1H-indol-3-yl)-2- | | | | | |
| | methyl-N-(1-pyridin-2-yl- | | | | | |
| | cyclohexylmethyl)-propionamide | | | | | |
| 53 | (S)-3-(1H-Indol-3-yl)-2-methyl-2-[2-(2- | 553, 67 | 95 | 1.5 | 1979 | IA |
| | nitro-phenyl)-ethanoylamino]-N-(1- | | | | | |
| | pyridin-2-yl-cyclohexylmethyl)- | | | | | |
| | propionamide | | | | | |
| 54 | (S)-3-(1H-Indol-3-yl)-2-[2-(2-methoxy- | 538, 70 | 100 | 1.57 | 1326 | 2479 |
| | phenyl)-ethanoylamino]-2-methyl-N- | | | | | |
| | (1-pyridin-2-yl-cyclohexylmethyl)- | | | | | |
| | propionamide | | | | | |

IA: IC50 > 10000 nM

### N-acyl Derivative of Intermediate VIb

### Example 55

### 1H-Indole-2-carboxylic acid ((S)-2-(1H-indol-3-yl)-1-[1-(5-methoxy-pyridin-2-yl)-cyclohexylmethyl]-carbamoyl}-1-methyl-ethyl)-amide

To a solution of 1-H-Indole-2-carboxylic acid (38 mg, 0.24 mmol), Intermediate VIb (100 mg, 0.19 mmol) and diisopropylethylamine (61 mg, 0.47 mmol) in DMF (5 mL) was added HBTU (90 mg, 0.24 mmol). The reaction mixture was stirred at room temperature for 16 h. The reaction mixture was concentrated under reduced pressure and the residue was diluted with EtOAc, washed with brine, dried (MgSO₄) and concentrated under reduced pressure. The residue was purified by column chromatography (60% EtOAc/heptane) to give Example 55 as an amorphous white solid (65 mg, 61%).

IR (film): 3285, 2931, 2855,1651, 1537, 1489, 1456, 1420, 1342,1310, 1267,1028, 908, 744 cm⁻¹;

NMR (CDCl₃): □=1.10-1.61 (11H, m), 1.95-2.04 (2H, m), 3.29-3.52 (4H, m), 3.43 (3H, s), 6.47 (1H,s), 6.86-6.90 (1H, m), 6.98-6.99 (2H, m), 7.09-7.42 (8H, m), 7.52-7.58 (2H, m), 7.73-7.74 (1H, m) 8.05 (1H, s), 9.11 (1H, s);

MS m/e (ES+): 564 (M⁺+H, 100%).

Binding studies of Example 55 to the bombesin receptors gave the following results (IC₅₀): BB₁: 11 nM, BB₂: 119 nM.

### Examples 56-79

### N-terminal Urethane Derivatives of Intermediate VIa

Scheme 3 describes the synthesis of urethane derivatives of Intermediate VIa:

Conversion of alcohol into 4-nitrophenyl carbonates

N-terminal urethane formation

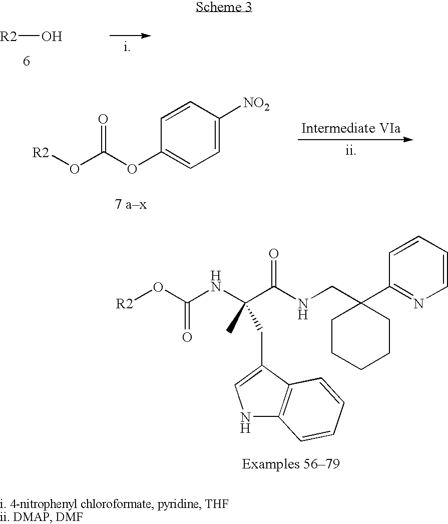

In scheme 3, R2 represents the rest of the intermediate (6). These intermediates (6) are listed in table 3.

To a stirred solution of alcohol 6 (10 mmol) and 4-nitrophenyl chloroformate (2.01 g, 10 mmol) in dichloromethane (50 mL) at 0° C. was added dropwise a solution of pyridine (0.81 mL, 10 mmol) in dichloromethane (10 mL). The reaction mixture was allowed to slowly warm to room temperature and was stirred at room temperature for 16 h. The solvent was removed under reduced pressure and the residue was taken up in EtOAc (50 mL) and was washed successively with 10% citric acid (2×30 mL), water (30 mL), sat. NaHCO₃ solution (2×50 mL) and brine (50 mL). The organic phase was dried (MgSO₄) and was concentrated under reduced pressure. The crude product was recrystallised from typically EtOAc, diethyl ether or heptane to give pure carbonate 7. The product was characterised by IR (see Table 2 for carbonate signals).

To carbonate 7 (0.21 mmol) was added DMF (0.4 mL) followed by 0.50 M DMAP in DMF (400 μL, 0.20 mmol) and 0.50 M Intermediate VIa in DMF (200 μL, 0.10 mmol). The solution was shaken on an orbital shaker at room temperature for 42 h. Water (1.0 mL) was added and the mixture was loaded onto a LC-18 SPE cartridge (0.5 g sorbent) and the cartridge was eluted with 25% methanol/water (3.4 mL) and methanol (4 mL). The methanol fraction was concentrated and purified by prep. HPLC (column: Phenomenex primesphere 10μ C18-HC 110A, 100×21.20 mm; mobile phase: methanol/water 10 to 100% gradient). The products were characterised and analysed by LCMS (column: 50×4.6 mm Prodigy ODSIII (5μ) column; mobile phase: acetonitrile/water (0.1% formic acid) 5 to 100% gradient over 2 min, held at 100% acetonitrile for 1 min; flow rate 4 mL/min; UV detection at 215 nm; mass spec: 150-900 Da full scan APCI+ centroid data).

The following products were made by the above method, with the starting material listed in Table 3 and gave the test results indicated in Table 4:

TABLE 3

| | | intermediate 7: |
|---|---|---|
| Example | intermediate 6 | IR (cm⁻¹) |
| 56 | Naphthalen-1-yl-methanol | 1754 |
| 57 | (3,4-Dimethoxy-phenyl)-methanol | 1754 |
| 58 | Naphthalen-2-yl-methanol | 1752 |
| 59 | Indan-2-ol | 1765 |
| 60 | (3,4-Dichloro-phenyl)-methanol | 1754 |
| 61 | (4-Methoxy-phenyl)-methanol | 1748 |
| 62 | (4-Chloro-phenyl)-methanol | 1761 |
| 63 | (2-Fluoro-phenyl)-methanol | 1752 |
| 64 | (2-Chloro-phenyl)-methanol | 1764 |
| 65 | (4-Nitro-phenyl)-methanol | 1761 |
| 66 | o-Tolyl-methanol | 1757 |
| 67 | (4-tert-Butyl-phenyl)-methanol | 1766 |
| 68 | (3-Nitro-phenyl)-methanol | 1769 |
| 69 | (2-Methoxy-phenyl)-methanol | 1766 |
| 70 | (4-Trifluoromethyl-phenyl)-methanol | 1763 |
| 71 | (3-Ethoxy-phenyl)-methanol | 1767 |
| 72 | 3-Hydroxymethyl-benzonitrile | 1769 |
| 73 | (2,4-Dichloro-phenyl)-methanol | 1768 |
| 74 | m-Tolyl-methanol | 1757 |
| 75 | (3-Phenoxy-phenyl)-methanol | 1766 |
| 76 | (3-Trifluoromethyl-phenyl)-methanol | 1770 |
| 77 | p-Tolyl-methanol | 1759 |
| 78 | (2,3-Dichloro-phenyl)-methanol | 1758 |
| 79 | Quinolin-6-yl-methanol | 1761 |

TABLE 4

| | | | | LCMS | | |
|---|---|---|---|---|---|---|
| | | | | Ret | BB₁ | BB₂ |
| Example | | | Purity | time | IC50 | IC50 |
| No | Product | MH⁺ | % | (min) | (nm) | (nm) |
| 56 | {(S)-2-(1H-Indol-3-yl)-1-methyl-1-[(1- | 574, 73 | 100 | 1.67 | 239 | IA |
| | pyridin-2-yl-cyclohexylmethyl)- | | | | | |
| | carbamoyl]-ethyl}-carbamic acid | | | | | |
| | naphthalen-1-ylmethyl ester | | | | | |
| 57 | {(S)-2-(1H-Indol-3-yl)-1-methyl-1-[(1- | 584, 72 | 95 | 1.41 | 1758 | IA |
| | pyridin-2-yl-cyclohexylmethyl)- | | | | | |
| | carbamoyl]-ethyl}-carbamic acid 3,4- | | | | | |
| | dimethoxy-benzyl ester | | | | | |
| 58 | {(S)-2-(1H-Indol-3-yl)-1-methyl-1-[(1- | 574, 73 | 100 | 1.67 | 1001 | IA |
| | pyridin-2-yl-cyclohexylmethyl)- | | | | | |
| | carbamoyl]-ethyl}-carbamic acid | | | | | |
| | naphthalen-2-ylmethyl ester | | | | | |
| 59 | {(S)-2-(1H-Indol-3-yl)-1-methyl-1-[(1- | 550, 71 | 91 | 1.59 | 955 | IA |
| | pyridin-2-yl-cyclohexylmethyl)- | | | | | |
| | carbamoyl]-ethyl}-carbamic acid | | | | | |
| | indan-2-yl ester | | | | | |
| 60 | {(S)-2-(1H-Indol-3-yl)-1-methyl-1-[(1- | 593, 56 | 93 | 1.73 | 202 | IA |
| | pyridin-2-yl-cyclohexylmethyl)- | | | | | |
| | carbamoyl]-ethyl}-carbamic acid 3,4- | | | | | |
| | dichloro-benzyl ester | | | | | |
| 61 | {(S)-2-(1H-Indol-3-yl)-1-methyl-1-[(1- | 554, 70 | 93 | 1.49 | 1610 | IA |
| | pyridin-2-yl-cyclohexylmethyl)- | | | | | |
| | carbamoyl]-ethyl}-carbamic acid 4- | | | | | |
| | methoxy-benzyl ester | | | | | |
| 62 | {(S)-2-(1H-Indol-3-yl)-1-methyl-1-[(1- | 559, 11 | 98 | 1.62 | 681 | IA |
| | pyridin-2-yl-cyclohexylmethyl)- | | | | | |
| | carbamoyl]-ethyl}-carbamic acid 4- | | | | | |
| | chloro-benzyl ester | | | | | |
| 63 | {(S)-2-(1H-Indol-3-yl)-1-methyl-1-[(1- | 542, 66 | 91 | 1.52 | 923 | IA |
| | pyridin-2-yl-cyclohexylmethyl)- | | | | | |
| | carbamoyl]-ethyl}-carbamic acid 2- | | | | | |
| | fluoro-benzyl ester | | | | | |
| 64 | {(S)-2-(1H-Indol-3-yl)-1-methyl-1-[(1- | 559, 11 | 89 | 1.62 | 624 | IA |
| | pyridin-2-yl-cyclohexylmethyl)- | | | | | |
| | carbamoyl]-ethyl}-carbamic acid 2- | | | | | |
| | chloro-benzyl ester | | | | | |
| 65 | {(S)-2-(1H-Indol-3-yl)-1-methyl-1-[(1- | 569, 67 | 97 | 1.51 | 41 | 463 |
| | pyridin-2-yl-cyclohexylmethyl)- | | | | | |
| | carbamoyl]-ethyl}-carbamic acid 4- | | | | | |
| | nitro-benzyl ester | | | | | |
| 66 | {(S)-2-(1H-Indol-3-yl)-1-methyl-1-[(1- | 538, 70 | 94 | 11.60 | 751 | IA |
| | pyridin-2-yl-cyclohexylmethyl)- | | | | | |
| | carbamoyl]-ethyl}-carbamic acid 2- | | | | | |
| | methyl-benzyl ester | | | | | |
| 67 | {(S)-2-(1H-Indol-3-yl)-1-methyl-1-[(1- | 580, 78 | 100 | 1.86 | 1986 | IA |
| | pyridin-2-yl-cyclohexylmethyl)- | | | | | |
| | carbamoyl]-ethyl}-carbamic acid 4- | | | | | |
| | tert-butyl-benzyl ester | | | | | |
| 68 | {(S)-2-(1H-Indol-3-yl)-1-methyl-1-[(1- | 569, 67 | 97 | 1.51 | 17 | 612 |
| | pyridin-2-yl-cyclohexylmethyl)- | | | | | |
| | carbamoyl]-ethyl}-carbamic acid 3- | | | | | |
| | nitro-benzyl ester | | | | | |
| 69 | {(S)-2-(1H-Indol-3-yl)-1-methyl-1-[(1- | 554, 70 | 96 | 1.52 | 818 | IA |
| | pyridin-2-yl-cyclohexylmethyl)- | | | | | |
| | carbamoyl]-ethyl}-carbamic acid 2- | | | | | |
| | methoxy-benzyl ester | | | | | |
| 70 | {(S)-2-(1H-Indol-3-yl)-1-methyl-1-[(1- | 592, 67 | 97 | 1.7 | 1102 | IA |
| | pyridin-2-yl-cyclohexylmethyl)- | | | | | |
| | carbamoyl]-ethyl}-carbamic acid 4- | | | | | |
| | trifluoromethyl-benzyl ester | | | | | |
| 71 | {(S)-2-(1H-Indol-3-yl)-1-methyl-1-[(1- | 568, 72 | 89 | 1.60 | 1065 | IA |
| | pyridin-2-yl-cyclohexylmethyl)- | | | | | |
| | carbamoyl]-ethyl}-carbamic acid 3- | | | | | |
| | ethoxy-benzyl ester | | | | | |
| 72 | {(S)-2-(1H-Indol-3-yl)-1-methyl-1-[(1- | 549, 68 | 99 | 1.43 | 85 | IA |
| | pyridin-2-yl-cyclohexylmethyl)- | | | | | |
| | carbamoyl]-ethyl}-carbamic acid 3- | | | | | |
| | cyano-benzyl ester | | | | | |
| 73 | {(S)-2-(1H-Indol-3-yl)-1-methyl-1-[(1- | 593, 56 | 95 | 1.78 | 450 | IA |
| | pyridin-2-yl-cyclohexylmethyl)- | | | | | |
| | carbamoyl]-ethyl}-carbamic acid 2,4- | | | | | |
| | dichloro-benzyl ester | | | | | |
| 74 | {(S)-2-(1H-Indol-3-yl)-1-methyl-1-[(1- | 538, 70 | 96 | 1.59 | 841 | IA |
| | pyridin-2-yl-cyclohexylmethyl)- | | | | | |
| | carbamoyl]-ethyl}-carbamic acid 3- | | | | | |
| | methyl-benzyl ester | | | | | |
| 75 | {(S)-2-(1H-Indol-3-yl)-1-methyl-1-[(1- | 616, 77 | 96 | 1.78 | 1350 | IA |
| | pyridin-2-yl-cyclohexylmethyl)- | | | | | |
| | carbamoyl]-ethyl}-carbamic acid 3- | | | | | |
| | phenoxy-benzyl ester | | | | | |
| 76 | {(S)-2-(1H-Indol-3-yl)-1-methyl-1-[(1- | 592, 67 | 96 | 1.67 | 182 | IA |
| | pyridin-2-yl-cyclohexylmethyl)- | | | | | |
| | carbamoyl]-ethyl}-carbamic acid 3- | | | | | |
| | trifluoromethyl-benzyl ester | | | | | |
| 77 | {(S)-2-(1H-Indol-3-yl)-1-methyl-1-[(1- | 538, 70 | 97 | 1.60 | 1084 | IA |
| | pyridin-2-yl-cyclohexylmethyl)- | | | | | |
| | carbamoyl]-ethyl}-carbamic acid 4- | | | | | |
| | methyl-benzyl ester | | | | | |
| 78 | {(S)-2-(1H-Indol-3-yl)-1-methyl-1-[(1- | 593, 56 | 94 | 1.73 | 152 | IA |
| | pyridin-2-yl-cyclohexylmethyl)- | | | | | |
| | carbamoyl]-ethyl}-carbamic acid 2,3- | | | | | |
| | dichloro-benzyl ester | | | | | |
| 79 | {(S)-2-(1H-Indol-3-yl)-1-methyl-1-[(1- | 575, 72 | 97 | 1.22 | 171 | IA |
| | pyridin-2-yl-cyclohexylmethyl)- | | | | | |
| | carbamoyl]-ethyl}-carbamic acid | | | | | |
| | quinolin-6-ylmethyl ester | | | | | |

### Examples 80-137

### N-terminal Sulfonamide Derivatives of Intermediate VIa

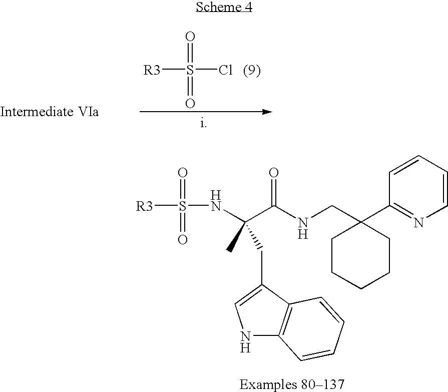

In scheme 4, R3 represents the rest of the intermediate (9). These intermediates (9) are listed in table 5

### N-sulfonamide Derivatives of Intermediate VIa

To sulfonyl chloride 9 (0.14 mmol) was added 0.143 M Intermediate VIa in DMF (700 μL, 0.10 mmol) followed by 300 μL of a solution containing a mixture of diisopropylethylamine (0.667 M in DMF, 0.20 mmol) and 4-dimethylaminopyridine (0.033 M in DMF, 0.01 mmol). The reaction mixture was shaken in an orbital shaker at 70° C. for 16 h. The crude reaction mixture was loaded onto a 5 g silica cartridge and the cartridge was eluted with EtOAc in heptane (30 to 100% gradient). Removal of the solvent under reduced pressure gave the sulfonamides (Examples 80-137). The purity of the sulfonamide was checked by LCMS. Those samples that were less than 95% pure were further purified by prep HPLC (column: YMC-Pack ODS-AM, 5 μm, 150×20 mm; mobile phase: acetonitrile/water 40 to 100% gradient). The products were characterised and analysed by LCMS (column: 150×4.6 mm Prodigy ODS3 (3μ) column; mobile phase: acetonitrile (0.085% TFA)/water (0.1% TFA) 20 to 100% gradient over 7 min, held at 100% acetonitrile (0.085% TFA) for 1 min; flow rate 1.5 mL/min; detection: diode array 200-300 nm; mass spec: 150-900 Da full scan APCI+ centroid data) (see Table 3).

The following examples were made by the above method, with the starting material listed in Table 5 and gave the test results indicated in Table 6:

TABLE 5

| Example | intermediate 9 |
|---|---|
| 80 | Phenyl-methanesulfonyl chloride |
| 81 | 4-Methyl-benzenesulfonyl chloride |
| 82 | 2-Chloro-benzenesulfonyl chloride |
| 83 | 2-Fluoro-benzenesulfonyl chloride |
| 84 | Naphthalene-1-sulfonyl chloride |
| 85 | 4-Chloro-benzenesulfonyl chloride |
| 86 | 5-Dimethylamino-naphthalene-1-sulfonyl chloride |
| 87 | Naphthalene-2-sulfonyl chloride |
| 88 | Thiophene-2-sulfonyl chloride |
| 89 | Quinoline-8-sulfonyl chloride |
| 90 | 3-Nitro-benzenesulfonyl chloride |
| 91 | 4-Fluoro-benzenesulfonyl chloride |
| 92 | 4-Nitro-benzenesulfonyl chloride |
| 93 | 3-Trifluoromethyl-benzenesulfonyl chloride |
| 94 | 3,4-Dichloro-benzenesulfonyl chloride |
| 95 | 3-Fluoro-benzenesulfonyl chloride |
| 96 | 4-Trifluoromethyl-benzenesulfonyl chloride |
| 97 | 5-Chloro-thiophene-2-sulfonyl chloride |
| 98 | 2-Trifluoromethyl-benzenesulfonyl chloride |
| 99 | 3-Chloro-benzenesulfonyl chloride |
| 100 | 3-Methyl-benzenesulfonyl chloride |
| 101 | 3,4-Dimethoxy-benzenesulfonyl chloride |
| 102 | 4-Cyano-benzenesulfonyl chloride |
| 103 | 2-Cyano-benzenesulfonyl chloride |
| 104 | 5-Chloro-1,3-dimethyl-1H-pyrazole-4-sulfonyl chloride |
| 105 | 3,5-Dimethyl-isoxazole-4-sulfonyl chloride |
| 106 | Benzo[1,2,5]thiadiazole-4-sulfonyl chloride |
| 107 | 1-Methyl-1H-imidazole-4-sulfonyl chloride |
| 108 | Benzo[1,2,5]oxadiazole-4-sulfonyl chloride |
| 109 | 3-Chlorosulfonyl-thiophene-2-carboxylic acid methyl ester |
| 110 | 5-Isoxazol-3-yl-thiophene-2-sulfonyl chloride |
| 111 | (2-Nitro-phenyl)-methanesulfonyl chloride |
| 112 | 3-Cyano-benzenesulfonyl chloride |
| 113 | 1,2-Dimethyl-1H-imidazole-4-sulfonyl chloride |
| 114 | 3-Methoxy-benzenesulfonyl chloride |
| 115 | 8-Nitro-naphthalene-1-sulfonyl chloride |
| 116 | 2-Chloro-5-nitro-benzenesulfonyl chloride |
| 117 | 2,4,6-Trichloro-benzenesulfonyl chloride |
| 118 | 4-Chloro-2-nitro-benzenesulfonyl chloride |
| 119 | 5-Benzenesulfonyl-thiophene-2-sulfonyl chloride |
| 120 | 4-Trifluoromethoxy-benzenesulfonyl chloride |
| 121 | 5-Methyl-2-phenoxy-benzenesulfonyl chloride |
| 122 | 2-p-Tolyloxy-benzenesulfonyl chloride |
| 123 | Biphenyl-2-sulfonyl chloride |
| 124 | 2-Chlorosulfonyl-benzoic acid methyl ester |
| 125 | 3-Chloro-4-fluoro-benzenesulfonyl chloride |
| 126 | 2,5-Dichloro-thiophene-3-sulfonyl chloride |
| 127 | 3-Chloro-4-methyl-benzenesulfonyl chloride |
| 128 | 2-Methoxy-4-methyl-benzenesulfonyl chloride |
| 129 | 5-Pyridin-2-yl-thiophene-2-sulfonyl chloride |
| 130 | 5-Bromo-6-chloro-pyridine-3-sulfonyl chloride |
| 131 | 2,4-Dinitro-benzenesulfonyl chloride |
| 132 | 4-Methanesulfonyl-benzenesulfonyl chloride |
| 133 | 4-tert-Butyl-benzenesulfonyl chloride |
| 134 | 2,4-Dichloro-5-methyl-benzenesulfonyl chloride |
| 135 | 2-Chloro-5-trifluoromethyl-benzenesulfonyl chloride |
| 136 | 2-Nitro-4-trifluoromethyl-benzenesulfonyl chloride |
| 137 | 4-Butyl-benzenesulfonyl chloride |

TABLE 6

| | | | | LCMS | | |
|---|---|---|---|---|---|---|
| | | | | Ret | BB₁ | BB₂ |
| Example | | | Purity | time | IC50 | IC50 |
| No | Product | MH⁺ | % | (min) | (nm) | (nm) |
| 80 | (S)-3-(1H-Indol-3-yl)-2-methyl-2- | 544, 72 | 100 | 4.64 | 186 | IA |
| | phenylmethanesulfonylamino-N-(1- | | | | | |
| | pyridin-2-yl-cyclohexylmethyl)- | | | | | |
| | propionamide | | | | | |
| 81 | (S)-3-(1H-Indol-3-yl)-2-methyl-N-(1- | 544, 72 | 100 | 4.74 | 557 | IA |
| | pyridin-2-yl-cyclohexylmethyl)-2- | | | | | |
| | (toluene-4-sulfonylamino)- | | | | | |
| | propionamide | | | | | |
| 82 | (S)-2-(2-Chloro- | 565, 14 | 100 | 4.71 | 257 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 83 | (S)-2-(2-Fluoro- | 548, 68 | 100 | 4.54 | 267 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 84 | (S)-3-(1H-Indol-3-yl)-2-methyl-2- | 580, 76 | 99 | 4.98 | 185 | 1576 |
| | (naphthalene-1-sulfonylamino)-N-(1- | | | | | |
| | pyridin-2-yl-cyclohexylmethyl)- | | | | | |
| | propionamide | | | | | |
| 85 | (S)-2-(4-Chloro- | 565, 14 | 97 | 4.89 | 373 | 4386 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 86 | (S)-2-(5-Dimethylamino- | 623, 82 | 100 | 4.39 | 1302 | IA |
| | naphthalene-1-sulfonylamino)-3-(1H- | | | | | |
| | indol-3-yl)-2-methyl-N-(1-pyridin-2-yl- | | | | | |
| | cyclohexylmethyl)-propionamide | | | | | |
| 87 | (S)-3-(1H-Indol-3-yl)-2-methyl-2- | 580, 76 | 100 | 5.01 | 322 | IA |
| | (naphthalene-2-sulfonylamino)-N-(1- | | | | | |
| | pyridin-2-yl-cyclohexylmethyl)- | | | | | |
| | propionamide | | | | | |
| 88 | (S)-3-(1H-Indol-3-yl)-2-methyl-N-(1- | 536, 72 | 99 | 4.39 | 232 | Ia |
| | pyridin-2-yl-cyclohexylmethyl)-2- | | | | | |
| | (thiophene-2-sulfonylamino)- | | | | | |
| | propionamide | | | | | |
| 89 | (S)-3-(1H-Indol-3-yl)-2-methyl-N-(1- | 581, 74 | 99 | 4.53 | 108 | IA |
| | pyridin-2-yl-cyclohexylmethyl)-2- | | | | | |
| | (quinoline-8-sulfonylamino)- | | | | | |
| | propionamide | | | | | |
| 90 | (S)-3-(1H-Indol-3-yl)-2-methyl-2-(3- | 575, 69 | 99 | 4.58 | 208 | 1960 |
| | nitro-benzenesulfonylamino)-N-(1- | | | | | |
| | pyridin-2-yl-cyclohexylmethyl)- | | | | | |
| | propionamide | | | | | |
| 91 | (S)-2-(4-Fluoro- | 548, 68 | 100 | 4.60 | 560 | 4165 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 92 | (S)-3-(1H-Indol-3-yl)-2-methyl-2-(4- | 575, 69 | 98 | 4.65 | 515 | IA |
| | nitro-benzenesulfonylamino)-N-(1- | | | | | |
| | pyridin-2-yl-cyclohexylmethyl)- | | | | | |
| | propionamide | | | | | |
| 93 | (S)-3-(1H-Indol-3-yl)-2-methyl-N-(1- | 599, 58 | 100 | 5.03 | 440 | 2246 |
| | pyridin-2-yl-cyclohexylmethyl)-2-(3- | | | | | |
| | trifluoromethyl- | | | | | |
| | benzenesulfonylamino)- | | | | | |
| | propionamide | | | | | |
| 94 | (S)-2-(3,4-Dichloro- | 599, 58 | 99 | 5.47 | 216 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 95 | (S)-2-(3-Fluoro- | 548, 68 | 100 | 4.65 | 407 | 2761 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 96 | (S)-3-(1H-Indol-3-yl)-2-methyl-N-(1- | 598, 69 | 95 | 5.31 | 553 | IA |
| | pyridin-2-yl-cyclohexylmethyl)-2-(4- | | | | | |
| | trifluoromethyl- | | | | | |
| | benzenesulfonylamino)- | | | | | |
| | propionamide | | | | | |
| 97 | (S)-2-(5-Chloro-thiophene-2- | 571, 17 | 99 | 4.94 | 404 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 98 | (S)-3-(1H-Indol-3-yl)-2-methyl-N-(1- | 598, 69 | 99 | 5.11 | 134 | — |
| | pyridin-2-yl-cyclohexylmethyl)-2-(2- | | | | | |
| | trifluoromethyl- | | | | | |
| | benzenesulfonylamino)- | | | | | |
| | propionamide | | | | | |
| 99 | (S)-2-(3-Chloro- | 565, 14 | 99 | 5.05 | 331 | 2687 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 100 | (S)-3-(1H-Indol-3-yl)-2-methyl-N-(1- | 544, 72 | 99 | 4.93 | 393 | 1019 |
| | pyridin-2-yl-cyclohexylmethyl)-2- | | | | | |
| | (toluene-3-sulfonylamino)- | | | | | |
| | propionamide | | | | | |
| 101 | (S)-2-(3,4-Dimethoxy- | 590, 75 | 98 | 4.50 | 608 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 102 | (S)-2-(4-Cyano- | 555, 70 | 99 | 4.61 | 766 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 103 | (S)-2-(2-Cyano- | 555, 70 | 97 | 4.62 | 408 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 104 | (S)-2-(5-Chloro-1,3-dimethyl-1H- | 583, 16 | 98 | 4.38 | 1252 | IA |
| | pyrazole-4-sulfonylamino)-3-(1H- | | | | | |
| | indol-3-yl)-2-methyl-N-(1-pyridin-2-yl- | | | | | |
| | cyclohexylmethyl)-propionamide | | | | | |
| 105 | (S)-2-(3,5-Dimethyl-isoxazole-4- | 549, 70 | 96 | 4.54 | 515 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 106 | (S)-2-(Benzo[1,2,5]thiadiazole-4- | 588, 76 | 97 | 4.67 | 256 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 107 | (S)-3-(1H-Indol-3-yl)-2-methyl-2-(1- | 534, 69 | 100 | 3.60 | 3667 | IA |
| | methyl-1H-imidazole-4- | | | | | |
| | sulfonylamino)-N-(1-pyridin-2-yl- | | | | | |
| | cyclohexylmethyl)-propionamide | | | | | |
| 108 | (S)-2-(Benzo[1,2,5]oxadiazole-4- | 572, 69 | 100 | 4.70 | 507 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 109 | 3-{(S)-2-(1H-Indol-3-yl)-1-methyl-1- | 594, 76 | 100 | 4.79 | 167 | IA |
| | [(1-pyridin-2-yl-cyclohexylmethyl)- | | | | | |
| | carbamoyl]-ethylsulfamoyl}- | | | | | |
| | thiophene-2-carboxylic acid methyl | | | | | |
| | ester | | | | | |
| 110 | (S)-3-(1H-Indol-3-yl)-2-(5-isoxazol-3- | 603, 77 | 98 | 4.60 | 534 | IA |
| | yl-thiophene-2-sulfonylamino)-2- | | | | | |
| | methyl-N-(1-pyridin-2-yl- | | | | | |
| | cyclohexylmethyl)-propionamide | | | | | |
| 111 | (S)-3-(1H-Indol-3-yl)-2-methyl-2-(2- | 589, 72 | 100 | 4.65 | 430 | IA |
| | nitro-phenylmethanesulfonylamino)- | | | | | |
| | N-(1-pyridin-2-yl-cyclohexylmethyl)- | | | | | |
| | propionamide | | | | | |
| 112 | (S)-2-(3-Cyano- | 555, 70 | 99 | 4.55 | 460 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 113 | (S)-2-(1,2-Dimethyl-1H-imidazole-4- | 548, 71 | 96 | 3.55 | 2482 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 114 | (S)-3-(1H-Indol-3-yl)-2-(3-methoxy- | 560, 72 | 99 | 4.75 | 295 | 3686 |
| | benzenesulfonylamino)-2-methyl-N- | | | | | |
| | (1-pyridin-2-yl-cyclohexylmethyl)- | | | | | |
| | propionamide | | | | | |
| 115 | (S)-3-(1H-Indol-3-yl)-2-methyl-2-(8- | 625, 75 | 99 | 4.89 | 177 | IA |
| | nitro-naphthalene-1-sulfonylamino)- | | | | | |
| | N-(1-pyridin-2-yl-cyclohexylmethyl)- | | | | | |
| | propionamide | | | | | |
| 116 | (S)-2-(2-Chloro-5-nitro- | 610, 14 | 96 | 5.00 | 374 | Ia |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 117 | (S)-3-(1H-Indol-3-yl)-2-methyl-N-(1- | 634, 03 | 100 | 5.45 | 215 | Ia |
| | pyridin-2-yl-cyclohexylmethyl)-2- | | | | | |
| | (2,4,6-trichloro- | | | | | |
| | benzenesulfonylamino)- | | | | | |
| | propionamide | | | | | |
| 118 | (S)-2-(4-Chloro-2-nitro- | 610, 14 | 100 | 5.13 | 513 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 119 | (S)-2-(5-Benzenesulfonyl-thiophene- | 676, 88 | 100 | 5.03 | 297 | IA |
| | 2-sulfonylamino)-3-(1H-indol-3-yl)-2- | | | | | |
| | methyl-N-(1-pyridin-2-yl- | | | | | |
| | cyclohexylmethyl)-propionamide | | | | | |
| 120 | (S)-3-(1H-Indol-3-yl)-2-methyl-N-(1- | 614, 69 | 99 | 5.35 | 635 | IA |
| | pyridin-2-yl-cyclohexylmethyl)-2-(4- | | | | | |
| | trifluoromethoxy- | | | | | |
| | benzenesulfonylamino)-propionamide | | | | | |
| 121 | (S)-3-(1H-Indol-3-yl)-2-methyl-2-(5- | 636, 82 | 97 | 5.79 | 76 | IA |
| | methyl-2-phenoxy- | | | | | |
| | benzenesulfonylamino)-N-(1-pyridin- | | | | | |
| | 2-yl-cyclohexylmethyl)-propionamide | | | | | |
| 122 | (S)-3-(1H-Indol-3-yl)-2-methyl-N-(1- | 636, 82 | 97 | 5.79 | 90 | IA |
| | pyridin-2-yl-cyclohexylmethyl)-2-(2-p- | | | | | |
| | tolyloxy-benzenesulfonylamino)- | | | | | |
| | propionamide | | | | | |
| 123 | (S)-2-(Biphenyl-2-sulfonylamino)-3- | 606, 79 | 97 | 5.52 | 166 | IA |
| | (1H-indol-3-yl)-2-methyl-N-(1-pyridin- | | | | | |
| | 2-yl-cyclohexylmethyl)-propionamide | | | | | |
| 124 | 2-{(S)-2-(1H-Indol-3-yl)-1-methyl-1- | 588, 73 | 99 | 4.84 | 242 | IA |
| | [(1-pyridin-2-yl-cyclohexylmethyl)- | | | | | |
| | carbamoyl]-ethylsulfamoyl}-benzoic | | | | | |
| | acid methyl ester | | | | | |
| 125 | (S)-2-(3-Chloro-4-fluoro- | 583, 13 | 95 | 5.12 | 284 | 1216 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 126 | (S)-2-(2,5-Dichloro-thiophene-3- | 605, 61 | 99 | 5.23 | 214 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 127 | (S)-2-(3-Chloro-4-methyl- | 579, 17 | 97 | 5.28 | 299 | 3939 |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 128 | (S)-3-(1H-Indol-3-yl)-2-(2-methoxy-4- | 574, 75 | 96 | 4.92 | 445 | IA |
| | methyl-benzenesulfonylamino)-2- | | | | | |
| | methyl-N-(1-pyridin-2-yl- | | | | | |
| | cyclohexylmethyl)-propionamide | | | | | |
| 129 | (S)-3-(1H-Indol-3-yl)-2-methyl-N-(1- | 613, 81 | 100 | 4.79 | 344 | IA |
| | pyridin-2-yl-cyclohexylmethyl)-2-(5- | | | | | |
| | pyridin-2-yl-thiophene-2- | | | | | |
| | sulfonylamino)-propionamide | | | | | |
| 130 | (S)-2-(5-Bromo-6-chloro-pyridine-3- | 645, 02 | 95 | 5.09 | 187 | IA |
| | sulfonylamino)-3-(1H-indol-3-yl)-2- | | | | | |
| | methyl-N-(1-pyridin-2-yl- | | | | | |
| | cyclohexylmethyl)-propionamide | | | | | |
| 131 | (S)-2-(2,4-Dinitro- | 620, 69 | 100 | 4.97 | 475 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 132 | (S)-3-(1H-Indol-3-yl)-2-(4- | 608, 78 | 98 | 4.20 | 1043 | IA |
| | methanesulfonyl- | | | | | |
| | benzenesulfonylamino)-2-methyl-N- | | | | | |
| | (1-pyridin-2-yl-cyclohexylmethyl)- | | | | | |
| | propionamide | | | | | |
| 133 | (S)-2-(4-tert-Butyl- | 586, 80 | 96 | 5.65 | 406 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 134 | (S)-2-(2,4-Dichloro-5-methyl- | 613, 61 | 97 | 5.64 | 172 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 135 | (S)-2-(2-Chloro-5-trifluoromethyl- | 633, 14 | 100 | 5.33 | 627 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |
| 136 | (S)-3-(1H-Indol-3-yl)-2-methyl-2-(2- | 643, 69 | 100 | 5.34 | 758 | IA |
| | nitro-4-trifluoromethyl- | | | | | |
| | benzenesulfonylamino)-N-(1-pyridin- | | | | | |
| | 2-yl-cyclohexylmethyl)-propionamide | | | | | |
| 137 | (S)-2-(4-Butyl- | 586, 80 | 96 | 5.84 | 492 | IA |
| | benzenesulfonylamino)-3-(1H-indol- | | | | | |
| | 3-yl)-2-methyl-N-(1-pyridin-2-yl- | | | | | |
| | cyclohexylmethyl)-propionamide | | | | | |

### BIOLOGICAL EXAMPLES

### EXAMPLE 138

### (Biological Example I)

### Method to Test Effect of Compounds of Formula (I) on Female Rat Sexual Proceptivity

The following method can be used to test the effect of compounds of this invention on the proceptivity of female rats. House in groups of 6 in a reversed lighting system of 12 h light:dark (lights off 7.00-19.00 h) ovariectomised adult female Sprague Dawley rats (180-200 g). Two weeks after ovariectomy the animals can be use for sexual activity tests. Adapt animals to the apparatus (in the absence of stimuli animals) for 10 min on 2 consecutive days prior to testing. Start the experiment at least 5 h into the dark period. Carry out tests in a circular arena of 90 cm diameter, surrounded by a 30 cm high wall. Two small cages with wire-mesh front (15×15 cm) are fixed into the wall such that the front of the cage is “flush” with the wall and the 2 cages are opposite each other. These will contain two stimuli animals: an intact sexually experienced male and a receptive female (ovariectomised, primed with 5 μg oestradiol benzoate dissolved in corn oil and injected subcutaneously 48 h before the test and with 0.5 mg of progesterone 4 h before the test). Sexually naïve test and control animals are used. Forty eight hours before the tests, both the test and control animals can be primed with 5 μg oestradiol benzoate. Test animals are treated with the compound(s) of formula (I) (30-100 mg/kg) dissolved in an appropriate vehicle and administered in a 1 ml/kg volume 1 h before each test. For animals used as positive controls, progesterone (0.5 mg/0.1 ml) can be dissolved in corn oil and administered subcutaneously (s.c.), 4 h before the test. Test and control animals are then introduced one at a time for 10-minute periods into the arena. During the 10-min test, the time that the test or positive control animal spent investigating each stimulus animal are noted. The arena should be thoroughly cleaned between animals. The position of the male/female stimuli boxes is randomised between animals, in order to avoid place preference. The difference in the percentage of time spent investigating male minus female can be calculated, out of the total time spent investigating stimuli animals. Analysis of this data will help determine if the compounds of formula (I) are beneficial in the treatment of sexual dysfunction.

### Example 139 (Biological Example II)

### Method to Test Effect of Compounds of Formula (I) on Female Rat Sexual Receptivity

The following method can be used to test the effect of compounds of this invention on the receptivity of female rats. House in groups of 6 in a reversed lighting system of 12 h light:dark (lights off 7.00-19.00 h) ovariectomised adult female Sprague Dawley rats (180-200 g). Two weeks after ovariectomy the animals can be use for sexual activity tests. The experiments should start at least 5 h into the dark period. The above compound of formula (I) can be dissolved in appropriate vehicle and administered. Quinelorane dihydrochloride (LY 163,502, 6.25 μg/kg) can be dissolved in water and administered s.c., as a positive control. Compounds can be administered in a 1-ml/kg volume. Forty eight hours before tests, the animals are primed with 5 μg oestradiol benzoate dissolved in corn oil and injected s.c. The females are then placed with a series of vigorous male rats and subjected to 10 mounts. The lordotic response of the animal is recorded and expressed as a percentage of the mounts (i.e. lordosis quotient, LQ). Treatment induced LQ=0-10% in most of the animals, can be considered non-receptive (NR). Animals showing higher LQ are excluded from the study. Each rat should be tested prior to administration of the compound of formula (I) and then tested similarly at 1 h and at 90 min post-injection of the above compound or quinelorane respectively. Analysis of this data will help determine if the compounds of formula (I) are beneficial in the treatment of sexual dysfunction.

1. A compound of formula (I) or a pharmaceutically acceptable salt thereof: 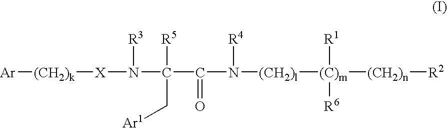
wherein:
k is 0, 1 or 2;
l is 0, 1, 2 or 3;
m is 0 or 1;
n is 0, 1 or 2;
X is —CO—, —OCO, —SO— or —SO₂—;
Ar is benzimidazolyl, benzofuryl, benzothiadiazolyl, benzothiazolyl, benzothienyl, benzopyrazinyl, benzotriazolyl, benzoxadiazolyl, furyl, imidazolyl, indanyl, indolyl, isoquinolyl, isoxazolyl, naphthyl, oxazolyl, phenyl, pyrazinyl, pyrazolyl, pyridyl, pyridazinyl, pyrimidyl, pyrrolyl, quinolyl, tetralinyl, tetrazolyl, thiazolyl, thienyl or triazolyl each unsubstituted or substituted with from 1 to 3 substituents selected from amino, acetyl, alkyl (straight chain or branched with from 1 to 6 carbon atoms), alkoxy, cyano, halogen, hydroxy, nitro, phenyl, pyridyl, pyrrolyl, isoxazolyl, phenoxy, tolyloxy, —CF₃, —OCF₃, —SO₂CF₃, —NHCONH₂, —CO₂H, —CH₂CO₂H, —CH₂CN, SO₂Me, SO₂NH₂, SO₂Ph, —(CH₂)_{q}NR⁷R⁸, —CONR⁹R¹⁰, and CO₂R¹¹, wherein q is 0, 1 or 2 and R⁷, R⁸, R⁹, R¹⁰, R¹¹ are each independently selected from hydrogen or straight or branched alkyl of up to 6 carbon atoms or cyclic alkyl of between 5 to 7 atoms which may contain 1 or 2 oxygen or nitrogen atoms or R⁷ and R⁸ or R⁹ and R¹⁰ together with the nitrogen atom to which they are linked can form a 5- to 7-membered aliphatic ring which may contain 1 or 2 oxygen or nitrogen atoms;
Ar¹ is independently selected from Ar and can also be pyridyl-N-oxide;
R¹ is hydrogen or straight or branched alkyl of up to 6 carbon atoms or cyclic alkyl of between 5 and 7 atoms which may contain 1 or 2 oxygen or nitrogen atoms;
R² is independently selected from Ar or is hydrogen, hydroxy, alkoxy, —NMe₂, —CONR¹²R¹³, 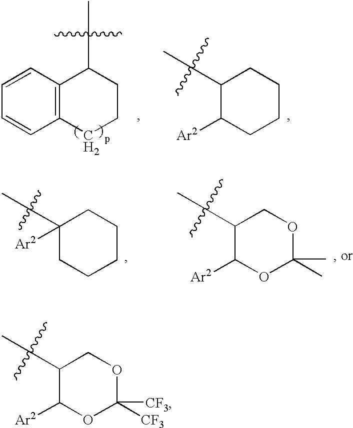
wherein p is 0, 1 or 2, Ar² is phenyl or pyridyl; and, R¹² and R¹³ are each independently selected from hydrogen, straight or branched alkyl of up to 6 carbon atoms or cyclic alkyl of between 5 and 7 carbon atoms;
R³, R⁴ and R⁵ are each independently selected from hydrogen and lower alkyl; and
R⁶ is hydrogen, methyl or forms with R¹ a ring of from 3 to 7 carbon atoms which can contain an oxygen or nitrogen atom, or R¹ and R⁶ can together be carbonyl;
provided that, when X is —OCO—, then 1 is 1, 2 or 3 and m is 1.

2. The compound of claim 1, wherein:
k is 0 or 1;
l is 1;
m is 0 or 1;
n is 0 or 1;
X is —CO—, —OCO—, or —SO₂—;
Ar is benzofuryl, furyl, indolyl, isoquinolyl, naphthyl, phenyl, pyridyl, quinolyl or thienyl each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ can form a ring of between 5 to 7 atoms which may contain 1 or 2 oxygen or nitrogen atoms, or R⁷ and R⁸ can be independently selected from hydrogen, straight or branched alkyl of up to 4 carbon atoms or cyclic alkyl of 5 carbon atoms;
Ar¹ is independently selected from Ar, and can also be pyridyl-N-oxide;
R¹ and R⁶ are cyclic alkyl of from 5 to 7 carbon atoms or R¹ and R⁶ together are carbonyl;
R² is independently selected from unsubstituted or substituted pyridyl or is hydrogen, hydroxy, alkoxy, —NMe₂, —CONR¹²R¹³ wherein R¹² and R¹³ are each independently selected from H and CH₃; and
R³, R⁴ and R⁵ are each independently selected from hydrogen and methyl.

3. The compound of claim 1, wherein:
l is 1;
m is 1;
n is 0;
R² is 2-pyridyl;
R⁶ forms a cyclohexyl with R¹.

4. A compound of formula (Ia): 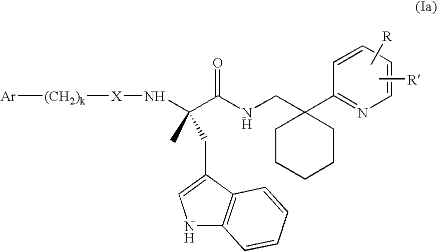
wherein Ar, k and X have the meanings given in claim 1 and the pyridine ring is optionally substituted by with 1 or 2 substituents, R and R′, independently selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ and R⁸ can be independently selected from hydrogen or cyclic alkyl of between 5 to 7 carbon atoms, or a pharmaceutically acceptable salt thereof.

5. The compound of claim 4, wherein Ar is benzofuryl, furyl, indolyl, isoquinolyl, naphthyl, phenyl, pyridyl, quinolyl or thienyl, each unsubstituted or substituted with 1 or 2 substituents selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷ or R⁸ can be a independently selected from hydrogen or cyclic alkyl of 5 carbon atoms anx X is —CO—, —OCO— or —SO₂.

6. The compound of claim 4 or 5, wherein X is —CO—.

7. The compound of claim 4 or 5, wherein X is —OCO—.

8. The compound of claim 4 or 5, wherein X is—and X is —SO₂—.

9. Any of the following compounds or a pharmaceutically acceptable salt thereof:
N-{(S)-2-(1H-indol-3-yl)-1-methyl-1-[(1-pyridin-2-yl-cyclohexylmethyl)-carbamoyl]-ethyl}-4-nitro-benzamide;
C-dimethylamino-N-{(S)-2-(1H-indol-3-yl)-1-methyl-1-[(1-pyridin-2-yl-cyclohexylmethyl)-carbamoyl]-ethyl}-benzamide;
1H-indole-2-carboxylic acid {(S)-2-(1H-indol-3-yl)-1-methyl-1-[(1-pyridin-2-yl-cyclohexylmethyl)-carbamoyl]-ethyl}-amide;
benzo[b]thiophene-2-carboxylic acid {(S)-2-(1H-indol-3-yl)-1-methyl-1-[(1-pyridin-2-yl-cyclohexylmethyl)-carbamoyl]-ethyl}-amide;
1H-indole-5-carboxylic acid {(S)-2-(1H-indol-3-yl)-1-methyl-1-[(1-pyridin-2-yl-cyclohexylmethyl)-carbamoyl]-ethyl}-amide; and
1H-indole-2-carboxylic acid ((S)-2-(1H-indol-3-yl)-1-{[1-(5-methoxy-pyridin-2-yl)-cyclohexylmethyl]-carbamoyl}-1-methyl-ethyl)-amide.

10. Any of the following compounds or a pharmaceutically acceptable salt thereof:
N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-4-methyl-benzamide;
4-chloro-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-4-methoxy-benzamide;
N-{(S)-2-(1H-indol-3-yl)-1-methyl-1-[(1-pyridin-2-yl-cyclohexylmethyl)-carbamoyl]-ethyl}-4-methanesulfonyl-benzamide;
3-cyano-N-{(S)-2-(1H-indol-3-yl)-1-methyl-1-[(1-pyridin-2-yl-cyclohexylmethyl)-carbamoyl]-ethyl}-benzamide;
3-chloro-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-3-methoxy-benzamide;
N-{(S)-2-(1H-indol-3-yl)-1-methyl-1-[(1-pyridin-2-yl-cyclohexylmethyl)-carbamoyl]-ethyl}-3-methanesulfonyl-benzamide;
dimethylamino-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-3-methyl-benzamide;
2-chloro-N-{(S)-2-(1H-indol-3-yl)-1-methyl-1-[(1-pyridin-2-yl-cyclohexylmethyl)-carbamoyl]-ethyl}-benzamide;
N-{(S)-2-(1H-indol-3-yl)-1-methyl-1-[(1-pyridin-2-yl-cyclohexylmethyl)-carbamoyl]-ethyl}-2-nitro-benzamide;
N-{(S)-2-(1H-indol-3-yl)-1-methyl-1-[(1-pyridin-2-yl-cyclohexylmethyl)-carbamoyl]-ethyl}-2-methoxy-benzamide;
N-{(S)-2-(1H-indol-3-yl)-1-methyl-1-[(1-pyridin-2-yl-cyclohexylmethyl)-carbamoyl]-ethyl}-2-methyl-benzamide;
2-fluoro-N-{(S)-2-(1H-indol-3-yl)-1-methyl-1-[(1-pyridin-2-yl-cyclohexylmethyl)-carbamoyl]-ethyl}-benzamide;
(S)-3-(1H-indol-3-yl)-2-methyl-N-(1-pyridin-2-yl-cyclohexylmethyl)-2-(2-p-tolyl-ethanoylamino)-propionamide;
(S)-3-(1H-indol-3-yl)-2-methyl-N-(1-pyridin-2-yl-cyclohexylmethyl)-2-(2-o-tolyl-ethanoylamino)-propionamide;
(S)-2-[2-(4-hydroxy-phenyl)-ethanoylamino]-3-(1H-indol-3-yl)-2-methyl-N-(1-pyridin-2-yl-cyclohexylmethyl)-propionamide;
(S)-2-[2-(3-hydroxy-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2-m-tolyl-ethanoylamino)-propionamide;
(S)-2-[2-(2-fluoro-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2-thiophen-3-yl-ethanoylamino)-propionamide;
N-{(S)-2-(1H-indol-3-yl)-1-methyl-1-[(1-pyridin-2-yl-cyclohexylmethyl)-carbamoyl]-ethyl}-isonicotinamide;
furan-3-carboxylic acid {(S)-2-(1H-indol-3-yl)-1-methyl-1-[(1-pyridin-2-yl-cyclohexylmethyl)-carbamoyl]-ethyl}-amide;
furan-2-carboxylic acid {(S)-2-(1H-indol-3-yl)-1-methyl-1-[(1-pyridin-2-yl-cyclohexylmethyl)-carbamoyl]-ethyl}-amide;
5-methyl-isoxazole-3-carboxylic acid {(S)-2-(1H-indol-3-yl)-1-methyl-1-[(1-pyridin-2-yl-cyclohexylmethyl)-carbamoyl]-ethyl}-amide;
1-methyl-1H-pyrrole-2-carboxylic acid {(S)-2-(1H-indol-3-yl)-1-methyl-1-[(1-pyridin-2-yl-cyclohexylmethyl)-carbamoyl]-ethyl}-amide;
thiophene-2-carboxylic acid {(S)-2-(1H-indol-3-yl)-1-methyl-1-[(1-pyridin-2-yl-cyclohexylmethyl)-carbamoyl]-ethyl}-amide;
thiophene-3-carboxylic acid {(S)-2-(1H-indol-3-yl)-1-methyl-1-[(1-pyridin-2-yl-cyclohexylmethyl)-carbamoyl]-ethyl}-amide;
1H-indole-6-carboxylic acid {(S)-2-(1H-indol-3-yl)-1-methyl-1-[(1-pyridin-2-yl-cyclohexylmethyl)-carbamoyl]-ethyl}-amide;
1H-indole-5-carboxylic acid {(S)-2-(1H-indol-3-yl)-1-methyl-1-[(1-pyridin-2-yl-cyclohexylmethyl)-carbamoyl]-ethyl}-amide;
1H-indole-4-carboxylic acid {(S)-2-(1H-indol-3-yl)-1-methyl-1-[(1-pyridin-2-yl-cyclohexylmethyl)-carbamoyl]-ethyl}-amide;
1H-indole-7-carboxylic acid {(S)-2-(1H-indol-3-yl)-1-methyl-1-[(1-pyridin-2-yl-cyclohexylmethyl)-carbamoyl]-ethyl}-amide;
1-methyl-1H-indole-2-carboxylic acid {(S)-2-(1H-indol-3-yl)-1-methyl-1-[(1-pyridin-2-yl-cyclohexylmethyl)-carbamoyl]-ethyl}-amide;
benzothiazole-6-carboxylic acid {(S)-2-(1H-indol-3-yl)-1-methyl-1-[(1-pyridin-2-yl-cyclohexylmethyl)-carbamoyl]-ethyl}-amide;
1H-benzotriazole-5-carboxylic acid {(S)-2-(1H-indol-3-yl)-1-methyl-1-[(1-pyridin-2-yl-cyclohexylmethyl)-carbamoyl]-ethyl}-amide;
3-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;
5-methyl-thiophene-2-carboxylic acid {(S)-2-(1H-indol-3-yl)-1-methyl-1-[(1-pyridin-2-yl-cyclohexylmethyl)-carbamoyl]-ethyl}-amide;
6-methyl-pyridine-2-carboxylic acid {(S)-2-(1H-indol-3-yl)-1-methyl-1-[(1-pyridin-2-yl-cyclohexylmethyl)-carbamoyl]-ethyl}-amide;
isoquinoline-3-carboxylic acid {(S)-2-(1H-indol-3-yl)-1-methyl-1-[(1-pyridin-2-yl-cyclohexylmethyl)-carbamoyl]-ethyl}-amide;
quinoxaline-2-carboxylic acid {(S)-2-(1H-indol-3-yl)-1-methyl-1-[(1-pyridin-2-yl-cyclohexylmethyl)-carbamoyl]-ethyl}-amide;
quinoline-8-carboxylic acid {(S)-2-(1H-indol-3-yl)-1-methyl-1-[(1-pyridiri-2-yl-cyclohexylmethyl)-carbamoyl]-ethyl}-amide;
5-phenyl-oxazole-4-carboxylic acid {(S)-2-(1H-indol-3-yl)-1-methyl-1-[(1-pyridin-2-yl-cyclohexylmethyl)-carbamoyl]-ethyl}-amide;
(S)-3-(1H-indol-3-yl)-2-[2-(4-methoxy-phenyl)-ethanoylamino]-2-methyl-N-(1-pyridin-2-yl-cyclohexylmethyl)-propionamide;
(S)-2-[2-(4-dimethylamino-phenyl)-ethanoylamino]-3-(1H-indol-3-yl)-2-methyl-N-(1-pyridin-2-yl-cyclohexylmethyl)-propionamide;
(S)-3-(1H-indol-3-yl)-2-methyl-2-[2-(2-nitro-phenyl)-ethanoylamino]-N-(1-pyridin-2-yl-cyclohexylmethyl)-propionamide;
(S)-3-(1H-indol-3-yl)-2-[2-(2-methoxy-phenyl)-ethanoylamino]-2-methyl-N-(1-pyridin-2-yl-cyclohexylmethyl)-propionamide; and
N-{(S)-2-(1H-indol-3-yl)-1-methyl-1-[(1-pyridin-2-yl-cyclohexylmethyl)-carbamoyl]-ethyl}-2-pyrrol-1-yl-benzamide.

11. Any of the following compounds and pharmaceutically acceptable salts thereof:
{(S)-2-(1H-indol-3-yl)-1-methyl-1-[(1-pyridin-2-yl-cyclohexylmethyl)-carbamoyl]-ethyl}-carbamic acid naphthalen-1-ylmethyl ester;
{(S)-2-(1H-indol-3-yl)-1-methyl-1-[(1-pyridin-2-yl-cyclohexylmethyl)-carbamoyl]-ethyl}-carbamic acid 3,4-dichloro-benzyl ester;
{(S)-2-(1H-indol-3-yl)-1-methyl-1-[(1-pyridin-2-yl-cyclohexylmethyl)-carbamoyl]-ethyl}-carbamic acid 3-nitro-benzyl ester;
{(S)-2-(1H-indol-3-yl)-1-methyl-1-[(1-pyridin-2-yl-cyclohexylmethyl)-carbamoyl]-ethyl}-carbamic acid 3-trifluoromethyl-benzyl ester;
{(S)-2-(1H-indol-3-yl)-1-methyl-1-[(1-pyridin-2-yl-cyclohexylmethyl)-carbamoyl]-ethyl}-carbamic acid quinolin-6-ylmethyl ester;
{(S)-2-(1H-indol-3-yl)-1-methyl-1-[(1-pyridin-2-yl-cyclohexylmethyl)-carbamoyl]-ethyl}-carbamic acid 4-nitro-benzyl ester; and
{(S)-2-(1H-indol-3-yl)-1-methyl-1-[(1-pyridin-2-yl-cyclohexylmethyl)-carbamoyl]-ethyl}-carbamic acid 3-cyano-benzyl ester.

12. Any of the following compounds and their pharmaceutically acceptable salts:
{(S)-2-(1H-indol-3-yl)-1-methyl-1-[(1-pyridin-2-yl-cyclohexylmethyl)-carbamoyl]-ethyl}-carbamic acid 3,4-dimethoxy-benzyl ester;
{(S)-2-(1H-indol-3-yl)-1-methyl-1-[(1-pyridin-2-yl-cyclohexylmethyl)-carbamoyl]-ethyl}-carbamic acid naphthalen-2-ylmethyl ester;
{(S)-2-(1H-indol-3-yl)-1-methyl-1-[(1-pyridin-2-yl-cyclohexylmethyl)-carbamoyl]-ethyl}-carbamic acid indan-2-yl ester;
{(S)-2,(1H-indol-3-yl)-1-methyl-1-[(1-pyridin-2-yl-cyclohexylmethyl)-carbamoyl]-ethyl}-carbamic acid 4-methoxy-benzyl ester;
{(S)-2-(1H-indol-3-yl)-1-methyl-1-[(1-pyridin-2-yl-cyclohexylmethyl)-carbamoyl]-ethyl}-carbamic acid 4-chloro-benzyl ester;
{(S)-2-(1H-indol-3-yl)-1-methyl-1-[(1-pyridin-2-yl-cyclohexylmethyl)-carbamoyl]-ethyl}-carbamic acid 2-fluoro-benzyl ester;
{(S)-2-(1H-indol-3-yl)-1-methyl-1-[(1-pyridin-2-yl-cyclohexylmethyl)-carbamoyl]-ethyl}-carbamic acid 2-chloro-benzyl ester;
{(S)-2-(1H-indol-3-yl)-1-methyl-1-[(1-pyridin-2-yl-cyclohexylmethyl)-carbamoyl]-ethyl}-carbamic acid 4-nitro-benzyl ester;
{(S)-2-(1H-indol-3-yl)-1-methyl-1-[(1-pyridin-2-yl-cyclohexylmethyl)-carbamoyl]-ethyl}-carbamic acid 2-methyl-benzyl ester;
{(S)-2-(1H-indol-3-yl)-1-methyl-1-[(1-pyridin-2-yl-cyclohexylmethyl)-carbamoyl]-ethyl}-carbamic acid 4-tert-butyl-benzyl ester;
{(S)-2-(1H-indol-3-yl)-1-methyl-1-[(1-pyridin-2-yl-cyclohexylmethyl)-carbamoyl]-ethyl}-carbamic acid 2-methoxy-benzyl ester;
{(S)-2-(1H-indol-3-yl)-1-methyl-1-[(1-pyridin-2-yl-cyclohexylmethyl)-carbamoyl]-ethyl}-carbamic acid 4-trifluoromethyl-benzyl ester;
{(S)-2-(1H-indol-3-yl)-1-methyl-1-[(1-pyridin-2-yl-cyclohexylmethyl)-carbamoyl]-ethyl}-carbamic acid 3-ethoxy-benzyl ester;
{(S)-2-(1H-indol-3-yl)-1-methyl-1-[(1-pyridin-2-yl-cyclohexylmethyl)-carbamoyl]-ethyl}-carbamic acid 3-cyano-benzyl ester;
{(S)-2-(1H-indol-3-yl)-1-methyl-1-[(1-pyridin-2-yl-cyclohexylmethyl)-carbamoyl]-ethyl}-carbamic acid 2,4-dichloro-benzyl ester;
{(S)-2-(1H-indol-3-yl)-1-methyl-1-[(1-pyridin-2-yl-cyclohexylmethyl)-carbamoyl]-ethyl}-carbamic acid 3-methyl-benzyl ester;
{(S)-2-(1H-indol-3-yl)-1-methyl-1-[(1-pyridin-2-yl-cyclohexylmethyl)-carbamoyl]-ethyl}-carbamic acid 3-phenoxy-benzyl ester;
{(S)-2-(1H-indol-3-yl)-1-methyl-1-[(1-pyridin-2-yl-cyclohexylmethyl)-carbamoyl]-ethyl}-carbamic acid 4-methyl-benzyl ester; and
{(S)-2-(1H-indol-3-yl)-1-methyl-1-[(1-pyridin-2-yl-cyclohexylmethyl)-carbamoyl]-ethyl}-carbamic acid 2,3-dichloro-benzyl ester.

13. Any of the following compounds and their pharmaceutically acceptable salts:
(S)-3-(1H-indol-3-yl)-2-methyl-2-phenylmethanesulfonylamino-N-(1-pyridin-2-yl-cyclohexylmethyl)-propionamide;
(S)-2-(2-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(naphthalene-1-sulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(quinoline-8-sulfonylamino)-propionamide;
(S)-3-(1H-indol-3-yl)-2-methyl-N-(1-pyridin-2-yl-cyclohexylmethyl)-2-(2-trifluoromethyl-benzenesulfonylamino)-propionamide;
(S)-2-(biphenyl-2-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(5-methyl-2-phenoxy-benzenesulfonyl-amino)-N-(1-pyridin-2-yl-cyclohexylmethyl)-propionamide; and
(S)-3-(1H-indol-3-yl)-2-methyl-N-(1-pyridin-2-yl-cyclohexylmethyl)-2-(2-p-tolyloxy-benzenesulfonylamino)-propionamide.

14. Any of the following compounds and their pharmaceutically acceptable salts:
(S)-3-(1H-indol-3-yl)-2-methyl-N-(1-pyridin-2-yl-cyclohexylmethyl)-2-(toluene-4-sulfonylamino)-propionamide;
(S)-3-(1H-indol-3-yl)-2-methanesulfonylamino-2-methyl-N-(1-pyridin-2-yl-cyclohexylmethyl)-propionamide;
(S)-2-(2-fluoro-benzenesulfonylamino)-3-(1H-indol-3-yl)-2-methyl-N-(1-pyridin-2-yl-cyclohexylmethyl)-propionamide;
(S)-2-(4-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2,2,2-trifluoro-ethanesulfonylamino)-propionamide;
(S)-2-(5-dimethylamino-naphthalene-1-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(naphthalene-2-sulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(thiophene-2-sulfonylamino)-propionamide;
(S)-3-(1H-indol-3-yl)-2-methyl-2-(3-nitro-benzenesulfonylamino)-N-(1-pyridin-2-yl-cyclohexylmethyl)-propionamide;
(S)-2-(4-fluoro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(4-nitro-benzenesulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(3-trifluoromethyl-benzenesulfonylamino)-propionamide;
(S)-2-(3,4-dichloro-benzenesulfonylamino)-3-(1H-indol-3-yl)-2-methyl-N-(1-pyridin-2-yl-cyclohexylmethyl)-propionamide;
(S)-2-(3-fluoro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(4-trifluoromethyl-benzenesulfonylamino)-propionamide;
(S)-2-(5-chloro-thiophene-2-sulfonylamino)-3-(1H-indol-3-yl)-2-methyl-N-(1-pyridin-2-yl-cyclohexylmethyl)-propionamide;
(S)-2-(3-chloro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(toluene-3-sulfonylamino)-propionamide;
(S)-2-(3,4-dimethoxy-benzenesulfonylamino)-3-(1H-indol-3-yl)-2-methyl-N-(1-pyridin-2-yl-cyclohexylmethyl)-propionamide;
(S)-2-(4-cyano-benzenesulfonylamino)-3-(1H-indol-3-yl)-2-methyl-N-(1-pyridin-2-yl-cyclohexylmethyl)-propionamide;
(S)-2-(2-cyano-benzenesulfonylamino)-3-(1H-indol-3-yl)-2-methyl-N-(1-pyridin-2-yl-cyclohexylmethyl)-propionamide;
(S)-2-(5-chloro-1,3-dimethyl-1H-pyrazole-4-sulfonylamino)-3-(1H-indol-3-yl )-2-methyl-N-(1-pyridin-2-yl-cyclohexylmethyl)-propionamide;
(S)-2-(3,5-dimethyl-isoxazole-4-sulfonylamino)-3-(1H-indol-3-yl)-2-methyl-N-(1-pyridin-2-yl-cyclohexylmethyl)-propionamide;
(S)-2-(benzo[1,2,5]thiadiazole-4-sulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(1-methyl-1H-imidazole-4-sulfonylamino)-N-(1-pyridin-2-yl-cyclohexylmethyl)-propionamide;
(S)-2-(benzo[1,2,5]oxadiazole-4-sulfonylamino)-3-(1H-indol-3-yl)-2-methyl-N-(1-pyridin-2-yl-cyclohexylmethyl)-propionamide;
3-{(S)-2-(1H-indol-3-yl)-1-methyl-1-[(1-pyridin-2-yl-cyclohexylmethyl)-carbamoyl]-ethylsulfamoyl}-thiophene-2-carboxylic acid methyl ester;
(S)-3-(1H-indol-3-yl)-2-(5-isoxazol-3-yl-thiophene-2-sulfonylamino)-2-methyl-N-(1-pyridin-2-yl-cyclohexylmethyl)-propionamide;
(S)-3-(1H-indol-3-yl)-2-methyl-2-(2-nitro-phenylmethanesulfonylamino)-N-(1-pyridin-2-yl-cyclohexylmethyl)-propionamide;
(S)-2-(3-cyano-benzenesulfonylamino)-3-(1H-indol-3-yl)-2-methyl-N-(1-pyridin-2-yl-cyclohexylmethyl)-propionamide;
(S)-2-(1,2-dimethyl-1H-imidazole-4-sulfonylamino)-3-(1H-indol-3-yl)-2-methyl-N-(1-pyridin-2-yl-cyclohexylmethyl)-propionamide;
(S)-3-(1H-indol-3-yl)-2-(3-methoxy-benzenesulfonylamino)-2-methyl-N-(1-pyridin-2-yl-cyclohexylmethyl)-propionamide;
(S)-3-(1H-indol-3-yl)-2-methyl-2-(8-nitro-naphthalene-1-sulfonylamino)-N-(1-pyridin-2-yl-cyclohexylmethyl)-propionamide;
(S)-2-(2-chloro-5-nitro-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2,4,6-trichloro-benzenesulfonylamino)-propionamide;
(S)-2-(4-chloro-2-nitro-benzenesulfonylamino)-3-(1H-indol-3-yl)-2-methyl-N-(1-pyridin-2-yl-cyclohexylmethyl)-propionamide;
(S)-2-(5-benzenesulfonyl-thiophene-2-sulfonylamino)-3-(1H-indol-3-yl)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(4-trifluoromethoxy-benzenesulfonylamino)-propionamide;
(S)-3-(1H-indol-3-yl)-2-methyl-2-(5-methyl-2-phenoxy-benzenesulfonylamino)-N-(1-pyridin-2-yl-cyclohexylmethyl)-propionamide;
(S)-3-(1H-indol-3-yl)-2-methyl-N-(1-pyridin-2-yl-cyclohexylmethyl)-2-(2-p-tolyloxy-benzenesulfonylamino)-propionamide;
2-{(S)-2-(1H-indol-3-yl)-1-methyl-1-[(1-pyridin-2-yl-cyclohexylmethyl)-carbamoyl]-ethylsulfamoyl}-benzoic acid methyl ester;
(S)-2-(3-chloro-4-fluoro-benzenesulfonylamino)-3-(1H-indol-3-yl)-2-methyl-N-(1-pyridin-2-yl-cyclohexylmethyl)-propionamide;
(S)-2-(2,5-dichloro-thiophene-3-sulfonylamino)-3-(1H-indol-3-yl)-2-methyl-N-(1-pyridin-2-yl-cyclohexylmethyl)-propionamide;
(S)-2-(3-chloro-4-methyl-benzenesulfonylamino)-3-(1H-indol-3-yl)-2-methyl-N-(1-pyridin-2-yl-cyclohexylmethyl)-propionamide;
(S)-3-(1H-indol-3-yl)-2-(2-methoxy-4-methyl-benzenesulfonylamino)-2-methyl-N-(1-pyridin-2-yl-cyclohexylmethyl)-propionamide;
(S)-3-(1H-indol-3-yl)-2-methyl-N-(1-pyridin-2-yl-cyclohexylmethyl)-2-(5-pyridin-2-yl-thiophene-2-sulfonylamino)-propionamide;
(S)-2-(5-bromo-6-chloro-pyridine-3-sulfonylamino)-3-(1H-indol-3-yl)-2-methyl-N-(1-pyridin-2-yl-cyclohexylmethyl)-propionamide;
(S)-2-(2,4-dinitro-benzenesulfonylamino)-3-(1H-indol-3-yl)-2-methyl-N-(1-pyridin-2-yl-cyclohexylmethyl)-propionamide;
(S)-3-(1H-indol-3-yl)-2-(4-methanesulfonyl-benzenesulfonylamino)-2-methyl-N-(1-pyridin-2-yl-cyclohexylmethyl)-propionamide;
(S)-2-(4-tert-butyl-benzenesulfonylamino)-3-(1H-indol-3-yl)-2-methyl-N-(1-pyridin-2-yl-cyclohexylmethyl)-propionamide;
(S)-2-(2,4-dichloro-5-methyl-benzenesulfonylamino)-3-(1H-indol-3-yl)-2-methyl-N-(1-pyridin-2-yl-cyclohexylmethyl)-propionamide;
(S)-2-(2-chloro-5-trifluoromethyl-benzenesulfonylamino)-3-(1H-indol-3-yl)-2-methyl-N-(1-pyridin-2-yl-cyclohexylmethyl)-propionamide;
(S)-3-(1H-indol-3-yl)-2-methyl-2-(2-nitro-4-trifluoromethyl-benzenesulfonyl-amino)-N-(1-pyridin-2-yl-cyclohexylmethyl)-propionamide; and
(S)-2-(4-butyl-benzenesulfonylamino)-3-(1H-indol-3-yl)-2-methyl-N-(1-pyridin-2-yl-cyclohexylmethyl)-propionamide.

15. A method for preparing a compound of claim 1, in which X is —CO— prepared by condensing an acid of the formula (II)
Ar—(CH₂)ₖ—COOH (II)
or a derivative thereof with an amine of the formula (III) 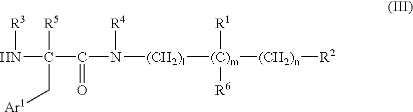
in an aprotic polar solvent in the presence of an appropriate catalyst, the values of the substituents Ar, Ar¹ and R¹ to R⁶ and the parameters k to n being as defined in claim 1, with reference to formula (I), and optionally converting the resulting product to a pharmaceutically acceptable salt.

16. The method of claim 15, wherein the condensation is carried out in O-benzotriazol-1-yl-N,N,N′,N′-tetramethyluronium hexafluorophosphate (HBTU) and N,N-diisopropyl-ethylamine (DIPEA).

17. A method for preparing a compound of claim 1, in which X is —OCO—, which comprises:
forming a carbonate from an alcohol of the formula (IV)
Ar—(CH₂)ₖ—OH (IV)
and reacting the carbonate with an amine of the formula (III) 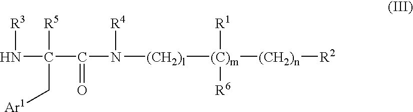
in an aprotic polar solvent in the presence of a base, the values of the substituents Ar, Ar¹ and R¹ to R⁶ and the parameters k to n being as defined above with reference to formula (I), and optionally converting the resulting product to a pharmaceutically acceptable salt.

18. The method of claim 17, wherein the compound of formula (IV) is reacted with 4-nitrophenyl chloroformate in dichloromethane in the presence of pyridine, and the resulting carbonate ester is reacted with the amine of formula (III) in dimethyl formamide in the presence of N,N-dimethyl-4-amino pyridine.

19. A method of preparing a compound of claim 1 in which X is —SO₂—, which comprises condensing a sulfonyl chloride of the formula (V)
Ar—(CH₂)ₖ—SO₂Cl (V)
with an amine of the formula (III) 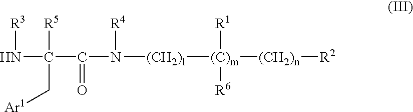
in an aprotic polar solvent in the presence of a base as catalyst, the values of the substituents Ar, Ar¹ and R¹ to R⁶ and the parameters k to n being as defined in claim 1, with reference to formula (I), and optionally converting the resulting product to a pharmaceutically acceptable salt.

20. The method of claim 19, wherein the condensation is carried out in dimethylformamide in the presence of N,N-diisopropylethylamine and N,N-dimethyl-4-aminopyridine.

21. The method of any of claims 15-20, wherein the amine of formula (III) is chiral (VI) 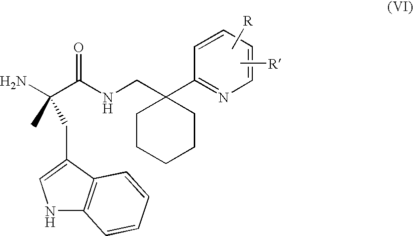
wherein the pyridine ring is optionally substituted by with 1 or 2 substituents R and R′ selected from alkoxy, cyano, halogen, nitro, phenyl, phenoxy, —CF₃, —(CH₂)_{q}NR⁷R⁸, wherein R⁷ and R⁸ together with the nitrogen atom to which they are linked can form a 5- to 7-membered aliphatic ring which may contain 1 or 2 oxygen or nitrogen atoms, or R⁷and R⁸ can be independently selected from hydrogen or cyclic alkyl of between 5 to 7 carbon atoms.

22. A compound of claim 21, wherein the compound has the formula (VIb)

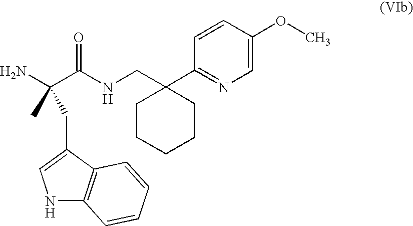

23. A salt of a compound of any of claims 1-14, wherein said salt is a hydrochloride, mesylate or sulfate.

24. A pharmaceutical composition comprising a therapeutically effective amount of a compound according to any of claims 1-14 in combination with a pharmaceutically acceptable carrier.

25. A method of antagonizing the effects of neuromedin B and/or gastrin-releasing peptide at bombesin receptors which comprises administering a compound according to any of claims 1-14 to a patient.

26. A method of treating sexual dysfunction in a male patient in need of said treatment comprising administering a therapeutically effective amount of a compound according to any one of claims 1-14.

27. A method of treating sexual dysfunction in a male patient, characterized by generalized unresponsiveness or ageing-related decline in sexual arousability, in need of said treatment comprising administering a therapeutically effective amount of a compound according to any one of claims 1-14.

28. Use of a compound of any of claims 1-14 in the manufacture of a medicament for preventing or treating sexual dysfunction in male patients.

29. Use of a compound of any of claims 1-14 in the manufacture of a medicament for preventing or treating sexual dysfunction in male patients characterized by generalized unresponsiveness or ageing-related decline in sexual arousability.

30. A method of treating sexual dysfunction in a female patient in need of said treatment comprising administering a therapeutically effective amount of a compound according to any of claims 1-14.

31. A method of treating sexual dysfunction characterized by generalized unresponsiveness or ageing-related decline in sexual arousability in a female patient in need of said treatment, comprising administering a therapeutically effective amount of a compound according to any of claims 1-14.

32. A method of treating sexual dysfunction in a female patient, characterized by hypoactive sexual desire disorders, sexual arousal disorders, orgasmic disorders or anorgasmy, or sexual pain disorders, in need of said treatment comprising administering a therapeutically effective amount of a compound according to any of claims 1-14.

33. Use of a compound of any of claims 1-14 in the manufacture of a medicament for preventing or treating sexual dysfunction in female patients in need of said treatment.

34. Use of a compound of any of claims 1-14 in the manufacture of a medicament for preventing or treating sexual dysfunction characterized by generalized unresponsiveness or ageing-related decline in sexual arousability in a female patient.

35. Use of a compound of any of claims 1-14 in the manufacture of a medicament for preventing or treating sexual dysfunction in female patients characterized by hypoactive sexual desire disorders, sexual arousal disorders, orgasmic disorders or anorgasmy, or sexual pain disorders.

36. A method of treating anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders, or pruritus in a patient in need of said treatment comprising administering a therapeutically effective amount of a compound according to any one of claims 1-14.

37. Use of any compound of any one of claims 1-14 in the manufacture of a medicament for preventing or treating anxiety and panic disorders, social phobia, depression, psychoses, sleeping disorders, memory impairment, pulmonary hypertension, lung repair and lung development disorders, cancer including prostate cancer and pancreatic cancer, hepatic porphyria, gastrointestinal secretory disturbances, gastrointestinal disorders including colitis, Crohn's disease and inflammatory bowel disease, emesis, anorexia, pain, seasonal affective disorders, feeding disorders and pruritus.

38. Use according to any of claims 28, 29, 33, 34, 35 and 37 wherein the medicament is adapted for oral administration.
